(19) United States
(12) Patent Application Publication
(10) Pub. No.: US 2023/0419480 A1
(43) Pub. Date: Dec. 28, 2023

(54) METHOD AND SYSTEM FOR PREDICTING CELLULAR AGING

(71) Applicants: New York Stem Cell Foundation, Inc., New York, NY (US); Memorial Sloan Kettering Cancer Center, New York, NY (US)

(72) Inventors: Bjarki Johannesson, New York, NY (US); Daniela Cornacchia, New York, NY (US); Bianca Migliori, New York, NY (US); Brodie Fischbacher, New York, NY (US)

(21) Appl. No.: 17/998,724

(22) PCT Filed: May 14, 2021
(86) PCT No.: PCT/US2021/032629

§ 371 Date: Nov. 14, 2022

Related U.S. Application Data
(60) Provisional application No. 63/024,762, filed on May 14, 2020.

(51) Int. Cl. G06T 7/00; G16H 50/20; G16H 50/70; G16H 10/40

(52) U.S. Cl. CPC G06T 7/0012; G16H 50/20; G16H 50/70; G16H 10/40; G06T 2207/10056; G06T 2207/20081; G06T 2207/30024

(57) ABSTRACT

The present disclosure provides automated methods and systems for implementing an aging analysis pipeline involving the training and deployment of a predictive model for predicting cellular ages of cells. Such a predictive model distinguishes between morphological cellular phenotypes e.g., morphological cellular phenotypes elucidated using Cell Paint, exhibited by cells of different ages. The predictive model is further useful for developing new cellular aging assays that include biomarkers that heavily contribute towards predictions of the predictive model. Furthermore, the predictive model is useful for screening drug candidates for their ability to alter or suppress age related phenotypes.

### CROSS REFERENCE TO RELATED APPLICATIONS

This application claims the benefit of and priority to U.S. Provisional Patent Application No. 63/024,762 filed May 14, 2020, the entire disclosure of which is hereby incorporated by reference in its entirety for all purposes.

### FIELD OF INVENTION

The present invention relates generally to the field of predictive analytics, and more specifically to automated methods and systems for predicting cellular age.

### BACKGROUND OF THE INVENTION

Age-related diseases are among the leading causes of mortality in the Western world. As the population ages, the prevalence and burden of these diseases increases, most of which lack optimal treatments. Common challenges in tackling age-dependent diseases include the complex, subtle, and interdependent nature of aging phenotypes, making it difficult to separate cause from consequence. Nonetheless, it is believed that a possible strategy for curbing the impact of age-related diseases would be identifying ways to intervene in the aging process itself. New, innovative approaches are needed to exploit this opportunity. Aging is likely a malleable process that can be modulated at the epigenetic level in different human cells and tissues. The advent of machine learning for recognizing often unexpected patterns in complex datasets where conventional analyses fail creates an unprecedented opportunity to define unique, complex aging phenotypes at the cellular level.

### SUMMARY OF THE INVENTION

Disclosed herein are methods and systems for performing high-content imaging of cells (e.g., human fibroblasts) from a large, age-diverse cohort to: a) discover complex aging phenotypes at the cellular level; b) develop cellular aging assays, and c) screen for drugs that can modulate aging phenotypes. To maximize the capture of unknown aging phenotypes, this unbiased approach analyzes morphological features, using advanced robotic automation procedures proven to reduce confounding variability. Machine learning algorithms (e.g., deep learning algorithms) can be applied to identify image features that distinguish cell age. Furthermore, known molecular markers of aging can be systematically evaluated and integrated to yield an optimized, age-tailored panel of cellular markers from which age-associated phenotypes are defined and quantified. These quantitative phenotypes can be used to screen a targeted, well-annotated library of epigenetically active molecules to yield candidate drugs with the potential to halt, hinder, or even reverse aging phenotypes. Notably, this approach enables the discovery of complex cellular phenotypes and chemical suppressors thereof in any disease of interest, representing a conceptual advance beyond current drug screening approaches that rely on single targets or functions.

Disclosed herein are concepts, approaches, and methodologies that, in various embodiments, can achieve the following:
A panel of well-characterized cells (e.g., fibroblasts) from an age-diverse cohort, including transcriptome-and epigenome-profiled lines,
Specific age-associated epigenetic changes identified in these lines that represent potential drug targets,
Automated, standardized procedures for cell (e.g., fibroblast) propagation and seeding as well as automated staining for high-content imaging, allowing multiple cell lines to be processed in parallel,
An innovative, unbiased, systematic, data-driven approach to identifying complex, age-related phenotypes, which have been notoriously challenging to define due to their subtle and interdependent nature,
An integrated cell painting and machine learning approach to define morphological phenotypes of differently aged cells, and
A drug screening assay that can screen for the effects of small molecule modifiers on cellular aging.

Disclosed herein is a method comprising: obtaining or having obtained a cell; capturing one or more images of the cell; and analyzing the one or more images using a predictive model to predict the cellular age of the cell, the predictive model trained to distinguish between morphological profiles of differently aged cells. In various embodiments, methods disclosed herein further comprise: prior to capturing one or more images of the cell, providing a perturbation to the cell; and subsequent to analyzing the one or more images, comparing the predicted cellular age of the cell to an age of the cell known before providing the perturbation; and based on the comparison, identifying the perturbation as having one of a directed aging effect, directed rejuvenation effect, or no effect. In various embodiments, analyzing the one or more images using a predictive model comprises separately applying the predictive model to each of the one or more images to predict cellular ages, wherein methods disclosed herein further comprise: evaluating performances of the predictive model across the predicted cellular ages; ranking the one or more images according to the evaluated performances of the predictive model across the predicted cellular ages; and selecting a set of biomarkers corresponding to the ranked channels for inclusion in a cellular aging assay.

In various embodiments, the predictive model is one of a neural network, random forest, or regression model. In various embodiments, each of the morphological profiles of differently aged cells comprise values of imaging features that define an age of a cell. In various embodiments, the imaging features comprise one or more of cell features or non-cell features. In various embodiments, the cell features comprise one or more of cellular shape, cellular size, cellular organelles, object-neighbors features, mass features, intensity features, quality features, texture features, and global features. In various embodiments, the non-cell features comprise well density features, background versus signal features, and percent of touching cells in a well. In various embodiments, the cell features are determined via fluorescently labeled biomarkers in the one or more images.

In various embodiments, the morphological profile is extracted from a layer of the neural network. In various embodiments, the morphological profile is an embedding representing a dimensionally reduced representation of values of the layer of the neural network. In various embodiments, the layer of the neural network is the penultimate layer of the neural network. In various embodiments, the cellular age of the cell predicted by the predictive model is a classification of at least two categories. In various embodiments, the at least two categories comprise a young cell category and an old cell category. In various embodiments, the at least two categories further comprises a middle-age cell category. In various embodiments, the young cell category corresponds to a subject that is less than 20 years old. In various embodiments, the old cell category corresponds to a subject that is greater than 60 years old. In various embodiments, the middle-age cell category corresponds to a subject that is between 20 years old and 60 years old.

In various embodiments, the cell is one of a stem cell, partially differentiated cell, or terminally differentiated cell. In various embodiments, the cell is a somatic cell. In various embodiments, the somatic cell is a fibroblast. In various embodiments, the predictive model is trained by: obtaining or having obtained a cell of a known cellular age; capturing one or more images of the cell of the known cellular age; and using the one or more images of the cell of the known cellular age, training the predictive model to distinguish between morphological profiles of differently aged cells. In various embodiments, the known cellular age of the cell serves as a reference ground truth for training the predictive model. In various embodiments, the cell of a known cellular age is one cell in an age-diverse cohort of cells.

In various embodiments, methods disclosed herein further comprise: prior to capturing the one or more images of the cell, staining the cell using one or more fluorescent dyes. In various embodiments, the one or more fluorescent dyes are Cell Paint dyes for staining one or more of a cell nucleus, cell nucleoli, plasma membrane, cytoplasmic RNA, endoplasmic reticulum, actin, Golgi apparatus, and mitochondria. In various embodiments, each of the one or more images correspond to a fluorescent channel. In various embodiments, the steps of obtaining the cell and capturing the one or more images of the cell are performed in a high-throughput format using an automated array.

Additionally disclosed herein is a non-transitory computer-readable medium comprising instructions that, when executed by a processor, cause the processor to: obtain or having obtained one or more images of a cell; and analyze the one or more images using a predictive model to predict the cellular age of the cell, the predictive model trained to distinguish between morphological profiles of differently aged cells. In various embodiments, the non-transitory computer-readable medium disclosed herein further comprise instructions that when executed by the processor cause the processor to: subsequent to analyzing the one or more images, compare the predicted cellular age of the cell to an age of the cell known before a perturbation was provided to the cell; and based on the comparison, identify the perturbation as having one of a directed aging effect, directed rejuvenation effect, or no effect. In various embodiments, the instructions that cause the processor to analyze the one or more images using a predictive model further comprises instructions that, when executed by the processor, cause the processor to separately apply the predictive model to each of the one or more images to predict cellular ages, wherein the instructions further comprise instructions that cause the processor to: evaluate performances of the predictive model across the predicted cellular ages; rank the one or more images according to the evaluated performances of the predictive model across the predicted cellular ages; and select a set of biomarkers corresponding to the ranked channels for inclusion in a cellular aging assay.

In various embodiments, the predictive model is one of a neural network, random forest, or regression model. In various embodiments, each of the morphological profiles of differently aged cells comprise values of imaging features that define an age of a cell. In various embodiments, the imaging features comprise one or more of cell features or non-cell features. In various embodiments, the cell features comprise one or more of cellular shape, cellular size, cellular organelles, object-neighbors features, mass features, intensity features, quality features, texture features, and global features. In various embodiments, the non-cell features comprise well density features, background versus signal features, and percent of touching cells in a well. In various embodiments, the cell features are determined via fluorescently labeled biomarkers in the one or more images.

In various embodiments, the morphological profile is extracted from a layer of the neural network. In various embodiments, the morphological profile is an embedding representing a dimensionally reduced representation of values of the layer of the neural network. In various embodiments, the layer of the neural network is the penultimate layer of the neural network. In various embodiments, the cellular age of the cell predicted by the predictive model is a classification of at least two categories. In various embodiments, the at least two categories comprise a young cell category and an old cell category. In various embodiments, the at least two categories further comprises a middle-age cell category. In various embodiments, the young cell category corresponds to a subject that is less than 20 years old. In various embodiments, the old cell category corresponds to a subject that is greater than 60 years old. In various embodiments, the middle-age cell category corresponds to a subject that is between 20 years old and 60 years old.

In various embodiments, the cell is one of a stem cell, partially differentiated cell, or terminally differentiated cell. In various embodiments, the cell is a somatic cell. In various embodiments, the somatic cell is a fibroblast. In various embodiments, the predictive model is trained by: obtaining or having obtained a cell of a known cellular age; capturing one or more images of the cell of the known cellular age; and using the one or more images of the cell of the known cellular age, training the predictive model to distinguish between morphological profiles of differently aged cells. In various embodiments, the known cellular age of the cell serves as a reference ground truth for training the predictive model. In various embodiments, the cell of the known cellular age is a cell in an age-diverse cohort of cells.

In various embodiments, the cell in the one or more images was previously stained using one or more fluorescent dyes. In various embodiments, the one or more fluorescent dyes are Cell Paint dyes for staining one or more of a cell nucleus, cell nucleoli, plasma membrane, cytoplasmic RNA, endoplasmic reticulum, actin, Golgi apparatus, and mitochondria. In various embodiments, each of the one or more images correspond to a fluorescent channel.

Additionally disclosed herein is a method comprising: obtaining or having obtained a cell; capturing one or more images of the cell; and analyzing imaging features derived from the one or more images using a predictive model to predict the cellular age of the cell, the predictive model trained to distinguish between morphological profiles of differently aged cells, wherein the imaging features comprise cell features and non-cell features, and wherein the morphological profiles of differently aged cells comprise values of imaging features that define an age of a cell. In various embodiments, methods disclosed herein further comprise: prior to capturing one or more images of the cell, providing a perturbation to the cell; and subsequent to analyzing the imaging features derived from the one or more images, comparing the predicted cellular age of the cell to an age of the cell known before providing the perturbation; and based on the comparison, identifying the perturbation as having one of a directed aging effect, directed rejuvenation effect, or no effect. In various embodiments, analyzing the imaging features derived from the one or more images using the predictive model comprises separately applying the predictive model to imaging features from each of the one or more images to predict cellular ages, wherein the method further comprises: evaluating performances of the predictive model across the predicted cellular ages; ranking the one or more images according to the evaluated performances of the predictive model across the predicted cellular ages; and selecting a set of biomarkers corresponding to the ranked channels for inclusion in a cellular aging assay. In various embodiments, the predictive model is one of a neural network, random forest, or regression model. In various embodiments, the cell features comprise one or more of cellular shape, cellular size, cellular organelles, object-neighbors features, mass features, intensity features, quality features, texture features, and global features. In various embodiments, the non-cell features comprise well density features, background versus signal features, and percent of touching cells in a well. In various embodiments, the cell features are determined via fluorescently labeled biomarkers in the one or more images.

In various embodiments, the morphological profile is extracted from a layer of the neural network. In various embodiments, the morphological profile is an embedding representing a dimensionally reduced representation of values of the layer of the neural network. In various embodiments, the layer of the neural network is the penultimate layer of the neural network. In various embodiments, the cellular age of the cell predicted by the predictive model is a classification of at least two categories. In various embodiments, the at least two categories comprise a young cell category and an old cell category. In various embodiments, the at least two categories further comprises a middle-age cell category. In various embodiments, the young cell category corresponds to a subject that is less than 20 years old. In various embodiments, the old cell category corresponds to a subject that is greater than 60 years old. In various embodiments, the middle-age cell category corresponds to a subject that is between 20 years old and 60 years old. In various embodiments, the cell is one of a stem cell, partially differentiated cell, or terminally differentiated cell. In various embodiments, the cell is a somatic cell. In various embodiments, the somatic cell is a fibroblast.

In various embodiments, the predictive model is trained by: obtaining or having obtained a cell of a known cellular age; capturing one or more images of the cell of the known cellular age; and using the one or more images of the cell of the known cellular age, training the predictive model to distinguish between morphological profiles of differently aged cells. In various embodiments, the known cellular age of the cell serves as a reference ground truth for training the predictive model. In various embodiments, the cell of a known cellular age is one cell in an age-diverse cohort of cells.

In various embodiments, methods disclosed herein further comprise: prior to capturing the one or more images of the cell, staining or having stained the cell using one or more fluorescent dyes. In various embodiments, the one or more fluorescent dyes are Cell Paint dyes for staining one or more of a cell nucleus, cell nucleoli, plasma membrane, cytoplasmic RNA, endoplasmic reticulum, actin, Golgi apparatus, and mitochondria. In various embodiments, each of the one or more images correspond to a fluorescent channel. In various embodiments, the steps of obtaining the cell and capturing the one or more images of the cell are performed in a high-throughput format using an automated array.

Additionally disclosed herein is a non-transitory computer readable medium comprising instructions that, when executed by a processor, cause the processor to perform any of the methods disclosed herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows a schematic cellular aging system for implementing an aging analysis pipeline, in accordance with an embodiment.

FIG. 2A is an example block diagram depicting the deployment of a predictive model, in accordance with an embodiment.

FIG. 2B is an example block diagram depicting the deployment of a predictive model, in accordance with a second embodiment.

FIG. 2C is an example structure of a predictive model, in accordance with an embodiment.

FIG. 3A is a flow process for training a predictive model for the aging analysis pipeline, in accordance with an embodiment.

FIG. 3B is a flow process for deploying a predictive model for the aging analysis pipeline, in accordance with an embodiment.

FIG. 4 is a flow process for developing a cellular aging assay by deploying a predictive model, in accordance with an embodiment.

FIG. 5 is a flow process for identifying modifiers of cellular age by deploying a predictive model, in accordance with an embodiment.

FIG. 6 depicts an example computing device for implementing system and methods described in reference to FIGS. 1-5.

FIG. 7A depicts an example aging analysis pipeline.

FIG. 7B depicts an example aging analysis pipeline in further detail.

FIG. 8A shows quantitative phenotypic differences across fibroblast cell lines of different ages.

FIG. 8B shows importance scores for various features of a random forest predictive model.

FIG. 8C demonstrates a matrix showing the accuracy of the random forest classifier when entire cell lines were removed from the training set in a single cell analysis.

FIG. 8D demonstrates a matrix showing the accuracy of the random forest classifier when entire cell lines were removed from the training set in a per-well analysis.

FIG. 9A depicts the predicted age determined by a regression model trained at the single-cell level using young, middle aged, and old cells.

FIG. 9B depicts the predicted age determined by a regression model trained at the single cell level using young and old cells.

FIG. 10 shows embedding distance versus actual cell line age distance.

FIG. 11A shows a heat map of top age-regulated genes.

FIG. 11B shows identification of differentially methylated regions in young and old fibroblasts using ERRBS.

FIG. 11C shows alignment of RNA-Seq data from fibroblasts and brain in collaboration with published RNA-Seq datasets from fibroblasts and brain identified novel robust aging biomarkers in both tissues.

FIG. 12 depicts an example drug screening pipeline.

### DETAILED DESCRIPTION

### Definitions

Terms used in the claims and specification are defined as set forth below unless otherwise specified.

As used in the specification and the appended claims, the singular forms “a,” “an” and “the” include plural referents unless the context clearly dictates otherwise.

The term “subject” encompasses a cell, tissue, or organism, human or non-human, whether male or female. In some embodiments, the term “subject” refers to a donor of a cell, such as a mammalian donor of more specifically a cell or a human donor of a cell.

The term “mammal” encompasses both humans and non-humans and includes but is not limited to humans, non-human primates, canines, felines, murines, bovines, equines, and porcines.

The phrase “morphological profile” refers to values of imaging features that define an age of a cell. In various embodiments, a morphological profile of a cell includes cell features (e.g., cell morphological features) including cellular shape and size as well as cell characteristics such as organelles including cell nucleus, cell nucleoli, plasma membrane, cytoplasmic RNA, endoplasmic reticulum, actin, Golgi apparatus, and mitochondria. In various embodiments, values of cell features are extracted from images of cells that have been labeled using fluorescently labeled biomarkers. Other cell features include object-neighbors features, mass features, intensity features, quality features, texture features, and global features (e.g., cell counts, cell distances). In various embodiments, a morphological profile of a cell includes values of non-cell features such as information about a well that the cell resides within (e.g., well density, background versus signal, percent of touching cells in the well). In various embodiments, a morphological profile of a cell includes values of both cell features and non-cell features, which define an age of a cell. In various embodiments, a predictive model is trained to distinguish between morphological profiles of differently aged cells.

The phrase “predictive model” refers to a machine learned model that distinguishes between morphological profiles of differently aged cells. Generally, a predictive model predicts the age of the cell based on the image features of a cell. Image features of the cell can be extracted from one or more images of the cell.

The phrase “obtaining a cell” encompasses obtaining a cell from a sample. The phrase also encompasses receiving a cell e.g., from a third party.

### Overview

In various embodiments, disclosed herein are methods and systems for performing high-throughput analysis of cells using an aging analysis pipeline that determines predicted ages of cells by implementing a predictive model trained to distinguish between morphological profiles of differently aged cells. A predictive model disclosed herein is useful for evaluating markers of cellular age, thereby enabling the development of new, more powerful cellular aging assays that incorporate more informative markers of cellular age. Furthermore, a predictive model disclosed herein is useful for performing high-throughput drug screens, thereby enabling the identification of modifiers of cellular age. Thus, modifiers of cellular age identified using the predictive model can be implemented for directed aging or directed rejuvenation.

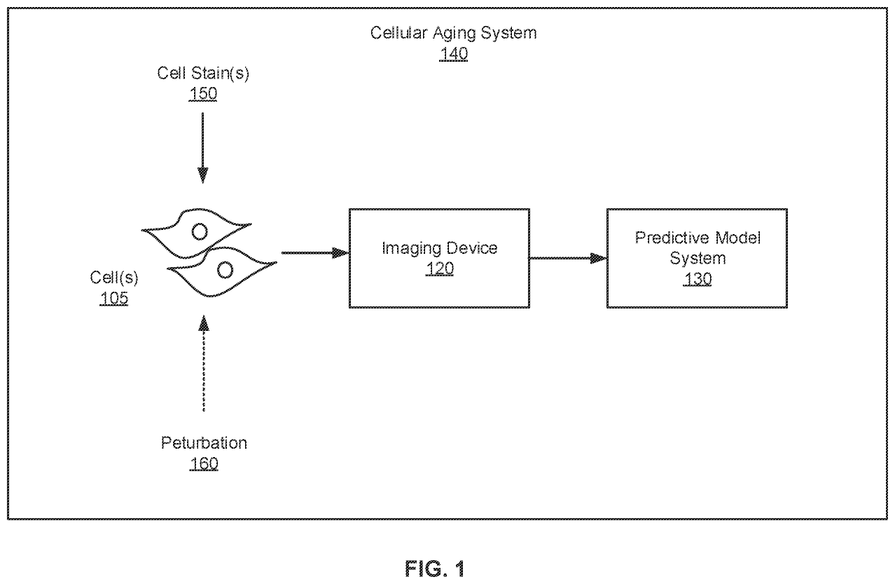

FIG. 1 shows an overall cellular aging system for implementing an aging analysis pipeline, in accordance with an embodiment. Generally, the cellular aging system 140 includes one or more cells 105 that are to be analyzed. In various embodiments, the cells 105 undergo a protocol for one or more cell stains 150. For example, cell stains 150 can be fluorescent stains for specific biomarkers of interest in the cells 105 (e.g., biomarkers of interest that can be informative for determining age of the cells 105). In various embodiments, the cells 105 can be exposed to a perturbation 160. Such a perturbation may have an effect on the age of the cell. In other embodiments, a perturbation 160 need not be applied to the cells 105.

The cellular aging system 140 includes an imaging device 120 that captures one or more images of the cells 105. The predictive model system 130 analyzes the one or more captured images of the cells 105. In various embodiments, the predictive model system 130 analyzes one or more captured images of multiple cells 105 to predict the age of the multiple cells 105. In various embodiments, the predictive model system 130 analyzes one or more captured images of a single cell to predict the age of the single cell.

In various embodiments, the predictive model system 130 analyzes one or more captured images of the cells 105, where different images are captured using different imaging channels. Therefore, different images include signal intensity indicating presence/absence of cell stains 150. Thus, the predictive model system 130 determines and selects cell stains that are informative for predicting the cell age of the cells 105. The selected cell stains can be included in a cellular aging assay for analysis of subsequent cells.

In various embodiments, the predictive model system 130 analyzes one or more captured images of the cells 105, where the cells 105 have been exposed to a perturbation 160. Thus, the predictive model system 130 can determine the age effects imparted by the perturbation 160. As one example, the predictive model system 130 can analyze a first set of images of cells captured before exposure to a perturbation 160 and a second set of images of the same cells captured after exposure to the perturbation 160. Thus, the change in the predicted ages can represent the aging effects of the perturbation 160.

Altogether, the cellular aging system 140 prepares cells 105 (e.g., exposes cells 105 to cell stains 150 and/or perturbation 160), captures images of the cells 105 using the imaging device 120, and predicts ages of the cells 105 using the predictive model system 130. In various embodiments, the cellular aging system 140 is a high-throughput system that processes cells 105 in a high-throughput manner such that large populations of cells are rapidly prepared and analyzed to predict cellular ages. The imaging device 120 may, through automated means, prepare cells (e.g., seed, culture, and/or treat cells), capture images from the cells 105, and provide the captured images to the predictive model system 130 for analysis. Additional description regarding the automated hardware and processes for handling cells are described below in Example 1. Further description regarding automated hardware and processes for handling cells are described in Paull, D., et al. Automated, high-throughput derivation, characterization and differentiation of induced pluripotent stem cells. Nat Methods 12, 885-892 (2015), which is incorporated by reference in its entirety.

### Predictive Model System

Generally, the predictive model system (e.g., predictive model system 130 described in FIG. 1) analyzes one or more images including cells that are captured by the imaging device 120. In various embodiments, the predictive model system analyzes images of cells for training a predictive model. In various embodiments, the predictive model system analyzes images of cells for deploying a predictive model to predict cellular age of a cell in the images.

In various embodiments, the images include fluorescent intensities of dyes that were previously used to stain certain components or aspects of the cells. In various embodiments, the images may have undergone Cell Paint staining and therefore, the images include fluorescent intensities of Cell Paint dyes that label cellular components (e.g., one or more of cell nucleus, cell nucleoli, plasma membrane, cytoplasmic RNA, endoplasmic reticulum, actin, Golgi apparatus, and mitochondria). Cell Paint is described in further detail in Bray et al., Cell Painting, a high-content image-based assay for morphological profiling using multiplexed fluorescent dyes. Nat. Protoc. 2016 September; 11(9): 1757-1774 as well as Schiff, L. et al., Deep Learning and automated Cell Painting reveal Parkinson's disease-specific signatures in primary patient fibroblasts, bioRxiv 2020.11.13.380576, each of which is hereby incorporated by reference in its entirety. In various embodiments, each image corresponds to a particular fluorescent channel (e.g., a fluorescent channel corresponding to a range of wavelengths). Therefore, each image can include fluorescent intensities arising from a single fluorescent dye with limited effect from other fluorescent dyes.

In various embodiments, prior to feeding the images to the predictive model (e.g., either for training the predictive model or for deploying the predictive model), the predictive model system performs image processing steps on the one or more images. Generally, the image processing steps are useful for ensuring that the predictive model can appropriately analyze the processed images. As one example, the predictive model system can perform a correction or a normalization over one or more images. For example, the predictive model system can perform a correction or normalization across one or more images to ensure that the images are comparable to one another. This ensures that extraneous factors do not negatively impact the training or deployment of the predictive model. An example correction can be an illumination correction which corrects for heterogeneities in the images that may arise from biases arising from the imaging device 120. Further description of illumination correction in Cell Paint images is described in Bray et al., Cell Painting, a high-content image-based assay for morphological profiling using multiplexed fluorescent dyes. Nat. Protoc. 2016 September; 11(9): 1757-1774, which is hereby incorporated by reference in its entirety.

In various embodiments, the image processing steps involve performing an image segmentation. For example, if an image includes multiple cells, the predictive model system performs an image segmentation such that resulting images each include a single cell. For example, if a raw image includes Y cells, the predictive model system may segment the image into Y different processed images, where each resulting image includes a single cell. In various embodiments, the predictive model system implements a nuclei segmentation algorithm to segment the images. Thus, a predictive model can subsequently analyze the processed images on a per-cell basis.

Generally, in analyzing one more images, the predictive model analyzes values of features of the images. The predictive model analyzes image features, which can include: cell features (e.g., cell morphological features) including cellular shape and size as well as cell characteristics such as organelles including cell nucleus, cell nucleoli, plasma membrane, cytoplasmic RNA, endoplasmic reticulum, actin, Golgi apparatus, and mitochondria. In various embodiments, values of cell features can be extracted from images of cells that have been labeled using fluorescently labeled biomarkers. Other cell features include object-neighbors features, mass features, intensity features, quality features, texture features, and global features. In various embodiments, image features include non-cell features such as information about a well that the cell resides within (e.g., well density, background versus signal, percent of touching cells in the well). In various embodiments, image features include CellProfiler features, examples of which are described in further detail in Carpenter, A. E., et al. CellProfiler: image analysis software for identifying and quantifying cell phenotypes. Genome Biol 7, R100 (2006), which is incorporated by reference in its entirety. In various embodiments, the values of features of the images are a part of a morphological profile of the cell. In various embodiments, to determine a predicted age of the cell, the predictive model compares the morphological profile of the cell (e.g., values of features of the images) extracted from an image to values of features for morphological profiles of other cells of known age (e.g., other cells of known age that were used during training of the predictive model). Further description of morphological profiles of cells is described herein.

In various embodiments, a feature extraction process can be performed to extract values of the aforementioned features from the images prior to implementing the predictive model. In various embodiments, the predictive model directly analyzes the images and extracts relevant feature values. For example, the predictive model may be a neural network that receives the images as input and performs the feature extraction.

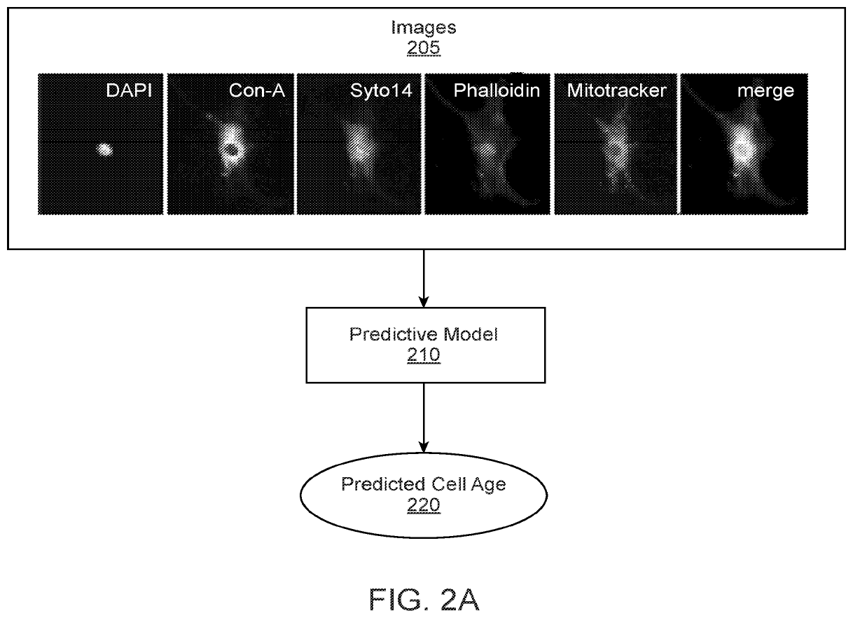

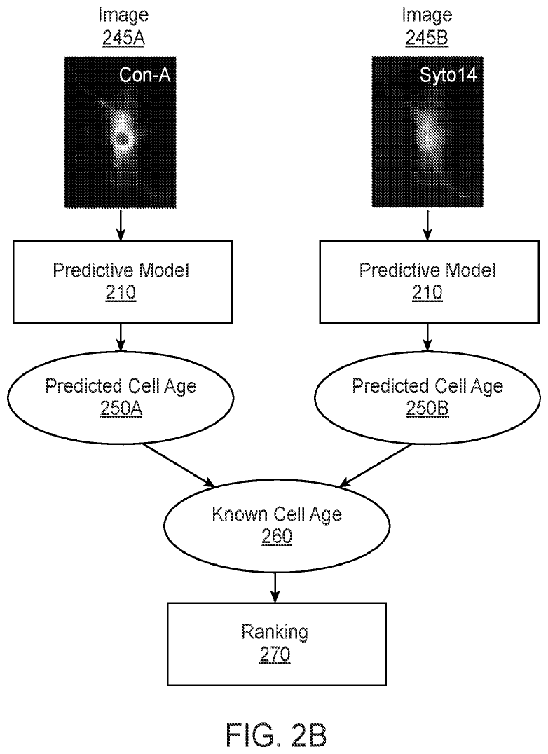

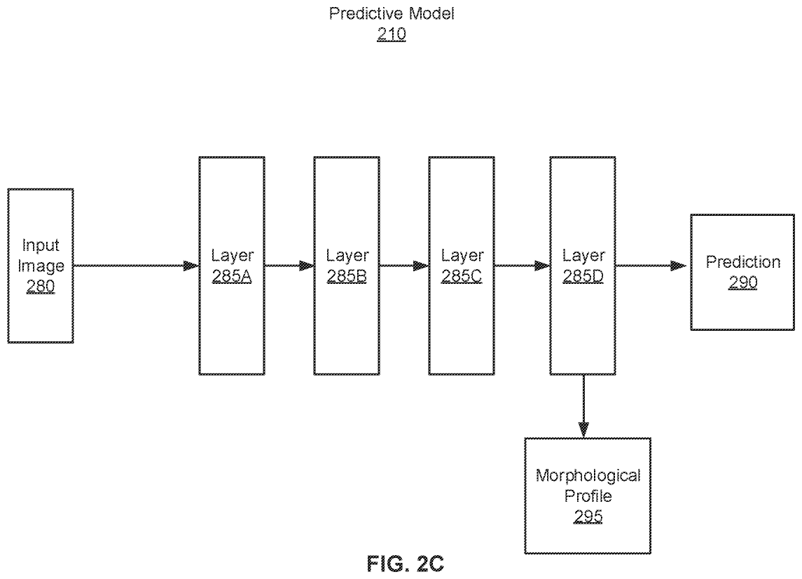

In various embodiments, the predictive model analyzes multiple images of a cell across different channels that have fluorescent intensities for different fluorescent dyes. Reference is now made to FIG. 2A, which is a block diagram that depicts the deployment of the predictive model, in accordance with an embodiment. FIG. 2A shows the multiple images 205 of a single cell. Here, each image 205 corresponds to a particular channel (e.g., fluorescent channel) which depicts fluorescent intensity for a fluorescent dye that has stained a marker of the cell. For example, as shown in FIG. 2A, a first image includes fluorescent intensity from a DAPI stain which shows the cell nucleus. A second image includes fluorescent intensity from a concanavalin A (Con-A) stain which shows the cell surface. A third image includes fluorescent intensity from a Syto14 stain which shows nucleic acids of the cell. A fourth image includes fluorescent intensity from a Phalloidin stain which shows actin filament of the cell. A fifth image includes fluorescent intensity from a Mitotracker stain which shows mitochondria of the cell. A sixth image includes the merged fluorescent intensities across the other images. Although FIG. 2A depicts six images with particular fluorescent dyes (e.g., images 205), in various embodiments, additional or fewer images with same or different fluorescent dyes may be employed.

As shown in FIG. 2A, the multiple images 205 can be provided as input to a predictive model 210. The predictive model 210 analyzes the multiple images 205 and determines a predicted cell age 220 for the cell in the images 205. The process can be repeated for other sets of images corresponding to other cells such that the predictive model 210 analyzes each other set of images to predict the age of the other cells.

In various embodiments, the predicted cell age 220 of the cell can be informative for determining an appropriate action for the cell. For example, predicted cell age 220 can serve as a quality control check that provides information as to whether the cell is of the expected age. For example, if the predicted cell age 220 indicates that the cell is older than an expected range, the cell can be discarded. As another example, if the predicted cell age 220 indicates that the cell is younger than an expected range, the cell can be further cultured until it of the appropriate age. As another example, if the predicted cell age 220 indicates that the cell is of the expected age, the cell can be used for subsequent analysis.

In various embodiments, the predicted cell age 220 of the cell can be compared to a previous cellular age of the cell. For example, the cell may have previously undergone a perturbation (e.g., by exposing to a drug), which may have had a directed aging or directed rejuvenation effect. Prior to the perturbation, the cell may have a previous cellular age. Thus, the previous cellular age of the cell is compared to the predicted cell age 220 to determine the effects of the perturbation. This is useful for identifying perturbations that are modifiers of cellular age.

In various embodiments, the predictive model analyzes individual images as opposed to multiple images. For example, each individual image includes the same cell, but corresponds to a different fluorescent channel. Thus, the predictive model can be separately deployed for individual images and predicts cellular age for the cell in each of the individual images. In other words, the predictive model predicts cellular age of a cell by only considering features of each single image. For each image (and corresponding fluorescent channel), the performance of the predictive model is evaluated based on the accuracy of the predictive model's prediction. For example, the predictive model may predict cellular age with higher accuracy when analyzing an image of a cell corresponding to a first fluorescent marker as compared to an image of a cell corresponding to a second fluorescent marker. Here, the accuracy of the predictive model can be determined by comparing each prediction to a known age of the cell. Thus, the first fluorescent marker may be more informative of cellular age as opposed to the second fluorescence. Thus, the first fluorescent marker can be ranked more highly than the second fluorescent marker. In various embodiments, the first fluorescent marker is selected for inclusion in a cellular aging assay due to its higher rank.

In various embodiments, more than two fluorescent markers are involved in the analysis. Therefore, in accordance with the above description, the different fluorescent markers can be ranked according to the performance of the predictive model when it analyzes images of each respective fluorescent marker. In various embodiments, at least a threshold number of markers can be selected for inclusion in the cellular aging assay. In various embodiments, the threshold number is two markers. In various embodiments, the threshold number is 3 markers, 4 markers, 5 markers, 6 markers, 7 markers, 8 markers, 9 markers, 10 markers, 11 markers, 12 markers, 13 markers, 14 markers, 15 markers, 16 markers, 17 markers, 18 markers, 19 markers, or 20 markers, or in any range between 2 markers and 20 markers. In particular embodiments, the threshold number is 5 markers. In particular embodiments, the threshold number is 10 markers. In particular embodiments, the threshold number is 20 markers.

Reference is now made to FIG. 2B, which is a block diagram that depicts the deployment of the predictive model, in accordance with a second embodiment. Here, individual image 245A and individual image 245B are separately provided as input to the predictive model 210. Although FIG. 2B depicts only two images (e.g., image 245A and image 245B corresponding to ConcavallinA and Syto14, respectively), in various embodiments, more than two images can be separately provided as input to the predicted model 210.

As shown in FIG. 2B, the predictive model 210 analyzes image 245A and determines predicted cell age 250A. Additionally, the predictive model 210 analyzes image 245B and determines predicted cell age 250B. Each of predicted cell age 250A and predicted cell age 250B is compared to the known cell age 260. For example, the comparison can include determining a difference between the known cell age 260 and each predicted cell age (e.g., predicted cell age 250A and predicted cell age 250B). The respective markers in the images (e.g., ConcavallinA and Syto14) can be including in a ranking 270 based on the comparison between the known cell age 260 and each respective predicted cell age (e.g., predicted cell age 250A and predicted cell age 250B). For example, if the difference between the known cell age 260 and predicted cell age 250A is smaller than the difference between the known cell age 260 and predicted cell age 260B, then the ConcavallinA stain is deemed more informative for predicting cell age in comparison to Syto14. Thus, ConcavallinA can be ranked higher than Syto14 in the ranking 270. Thus, the higher ranked marker (e.g., ConcavallinA) can be selected for inclusion in a cellular aging assay based on its rank in the ranking 270.

### Predictive Model

Generally, the predictive model analyzes an image with one or more cells or analyzes features extracted from an image with one or more cells. As a result of the analysis, the predictive model outputs a prediction of the age of the one or more cells in the image. In various embodiments, the predictive model can be any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). In various embodiments, the predictive model comprises a dimensionality reduction component for visualizing data, the dimensionality reduction component comprising any of a principal component analysis (PCA) component or a T-distributed Stochastic Neighbor Embedding (TSNe). In particular embodiments, the predictive model is a neural network. In particular embodiments, the predictive model is a random forest. In particular embodiments, the predictive model is a regression model.

In various embodiments, the predictive model includes one or more parameters, such as hyperparameters and/or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, variables and threshold for splitting nodes in a random forest, support vectors in a support vector machine, and coefficients in a regression model. The model parameters of the predictive model are trained (e.g., adjusted) using the training data to improve the predictive power of the predictive model.

In various embodiments, the predictive model outputs a classification of an age of a cell. In various embodiments, the predictive model outputs one of two possible classifications of an age of a cell. For example, the predictive model classifies a cell as either a young cell or an old cell. In various embodiments, the predictive model outputs one of three possible classifications of an age of a cell. For example, the predictive model classifies a cell as a young cell, a middle-aged cell, or an old cell. In one scenario, a young cell can represent a cell from a young subject who is less than 20 years old. In one scenario, a young cell can represent a cell from a young subject who is less than 15 years old. In one scenario, a young cell can represent a cell from a young subject who is less than 10 years old. In one scenario, a middle-aged cell can represent a cell from a middle-aged subject who is between 20 years old and 60 years old. In one scenario, a middle-aged cell can represent a cell from a middle-aged subject who is between 10 years old and 70 years old. In one scenario, a middle-aged cell can represent a cell from a middle-aged subject who is between 15 years old and 65 years old. In one scenario, an old cell can represent a cell from an old subject who is greater than 60 years old. In one scenario, an old cell can represent a cell from an old subject who is greater than 65 years old. In one scenario, an old cell can represent a cell from an old subject who is greater than 70 years old.

In various embodiments, the predictive model outputs a classification from a plurality of possible classifications. In various embodiments, the possible classifications can be a specific age. For example, the possible classifications can be X years old, where X is any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In various embodiments, the possible classifications can be a range of ages. For example, a range of ages can be 2 year ranges. For example, a range of ages can be 3 year ranges. For example, a range of ages can be 4 year ranges. For example, a range of ages can be 5 year ranges. For example, a range of ages can be 10 year ranges. For example, a range of ages can be 20 year ranges. For example, a range of ages can be 30 year ranges. For example, a range of ages can be 40 year ranges. For example, a range of ages can be 50 year ranges. In particular embodiments, the range of ages are 5 year ranges and thus, classifications can include one or more of: 0-5 years old, 5-10 years old, 10-15 years old, 15-20 years old, 20-25 years old, 25-30 years old, 30-35 years old, 35-40 years old, 40-45 years old, 45-50 years old, 50-55 years old, 55-60 years old, 60-65 years old, 70-75 years old, 75-80 years old, 80-85 years old, 85-90 years old, 90-95 years old, or 95-100 years old. In particular embodiments, the range of ages are 10 year ranges and thus, classifications can include one or more of: 0-10 years old, 10-20 years old, 20-30 years old, 30-40 years old, 40-50 years old, 50-60 years old, 60-70 years old, 70-80 years old, 80-90 years old, or 90-100 years old. In particular embodiments, the range of ages are 20 year ranges and thus, classifications can include one or more of: 0-20 years old, 20-40 years old, 40-60 years old, 60-80 years old, or 80-100 years old.

The predictive model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, gradient descent, and dimensionality reduction techniques such as manifold learning, principal component analysis, factor analysis, autoencoder regularization, and independent component analysis, or combinations thereof. In particular embodiments, the predictive model is trained using a deep learning algorithm. In particular embodiments, the predictive model is trained using a random forest algorithm. In particular embodiments, the predictive model is trained using a linear regression algorithm. In various embodiments, the predictive model is trained using supervised learning algorithms, unsupervised learning algorithms, semi-supervised learning algorithms (e.g., partial supervision), weak supervision, transfer, multi-task learning, or any combination thereof. In particular embodiments, the predictive model is trained using a weak supervision learning algorithm.

In various embodiments, the predictive model is trained to improve its ability to predict the age of a cell using training data that include reference ground truth values. For example, a reference ground truth value can be a known age of a cell. In a training iteration, the predictive model analyzes images acquired from the cell and determines a predicted age of the cell. The predicted age of the cell can be compared against the reference ground truth value (e.g., known age of the cell) and the predictive model is tuned to improve the prediction accuracy. For example, the parameters of the predictive model are adjusted such that the predictive model's prediction of the age of the cell is improved. In particular embodiments, the predictive model is a neural network and therefore, the weights associated with nodes in one or more layers of the neural network are adjusted to improve the accuracy of the predictive model's predictions. In various embodiments, the parameters of the neural network are trained using backpropagation to minimize a loss function. Altogether, over numerous training iterations across different cells, the predictive model is trained to improve its prediction of cell ages across the different cells.

In various embodiments, the predictive model is trained using weak supervision, given the limited available reference ground truths. For example, the predictive model may be trained to predict a cellular age of a cell across a full range (e.g., 0-100 years). However, the training data may be labeled with reference ground truths that only span a portion of that range. In various embodiments, the predictive model is trained on images labeled as either young or old. For example, the predictive model may be trained on images labeled as less than 10 years old (e.g., young) or greater than 70 years old (e.g., old). For example, the predictive model may be trained on images labeled as less than 20 years old (e.g., young) or greater than 60 years old (e.g., old). Thus, the predictive model can learn to predict ages of cells (e.g., ages between 10 and 70 years old or ages between 20 and 60 years old) even though it has not seen cells within that age range.

In various embodiments, a trained predictive model includes a plurality of morphological profiles that define cells of different ages. In various embodiments, a morphological profile for a cell of a particular age refers to a combination of values of features that define the cell of the particular age. For example, a morphological profile for a cell of a particular age may be a feature vector including values of features that are informative for defining the cell of the particular age. Thus, a second morphological profile for a cell of a different age can be a second feature vector including different values of the features that are informative for defining the cell of the different age.

In various embodiments, a morphological profile of a cell includes image features that are extracted from one or more images of the cell. Image features can include cell features (e.g., cell morphological features) including cellular shape and size as well as cell characteristics such as organelles including cell nucleus, cell nucleoli, plasma membrane, cytoplasmic RNA, endoplasmic reticulum, actin, Golgi apparatus, and mitochondria. In various embodiments, values of cell features can be extracted from images of cells that have been labeled using fluorescently labeled biomarkers. Other cell features include object-neighbors features, mass features, intensity features, quality features, texture features, and global features. In various embodiments, image features include non-cell features such as information about a well that the cell resides within (e.g., well density, background versus signal, percent of touching cells in the well).

In various embodiments, a morphological profile for a cell can include a representation of the aforementioned image features (e.g., cell features or non-cell features). For example, the predictive model can be a neural network and therefore, the morphological profile can be an embedding that is a representation of the aforementioned features. In various embodiments, the morphological profile is extracted from a layer of the neural network. As one example, the morphological profile for a cell can be extracted from the penultimate layer of the neural network. As one example, the morphological profile for a cell can be extracted from the third to last layer of the neural network. In this context, the representation of the aforementioned features refers to the values of features that have at least undergone transformations through the preceding layers of the neural network. In various embodiments, an embedding is a dimensionally reduced representation of values in a layer. Thus, an embedding can be used comparatively by calculating the Euclidean distance between the embedding and other embeddings of cells of known age as a measure of phenotypic distance.

Reference is now made to FIG. 2C, which depicts an example structure of a predictive model, in accordance with an embodiment. Here, the input image 280 is provided as input to a first layer 285A of the neural network. For example, the input image 280 can be structured as an input vector and provided to nodes of the first layer 285A. The first layer 285A transforms the input values and propagates the values through the subsequent layers 285B, 285C, and 285D. The predictive model 210 may determine a prediction 290 (e.g., predicted cellular age) based on the values in the layer 285D. In various embodiments, the layer 285D can represent the morphological profile 295 of the cell and can be a representation of the aforementioned features of the cell (e.g., cell features, non-cell features, or other example features). In various embodiments, the morphological profile 295 of the cell can be compared to morphological profiles of cells of known age. This can guide the prediction 290 determined by the predictive model 210. For example, if the morphological profile 295 of the cell is similar to a morphological profile of a cell of known age, then the predictive model 210 can predict that the cell is also of the known age.

Put more generally, in predicting the age of a cell, the predictive model can compare the values of features of the cell (or a representation of the features) to values of features (or a representation of the features) of one or more morphological profiles of cells of known age. For example, if the values of features (or a representation of the features) of the cell are closer to values of features (or a representation of the features) of a first morphological profile in comparison to values of features (or a representation of the features) of a second morphological profile, the predictive model can predict that the age of the cell is the cellular age corresponding to the first morphological profile.

### Methods for Determining Cellular Age

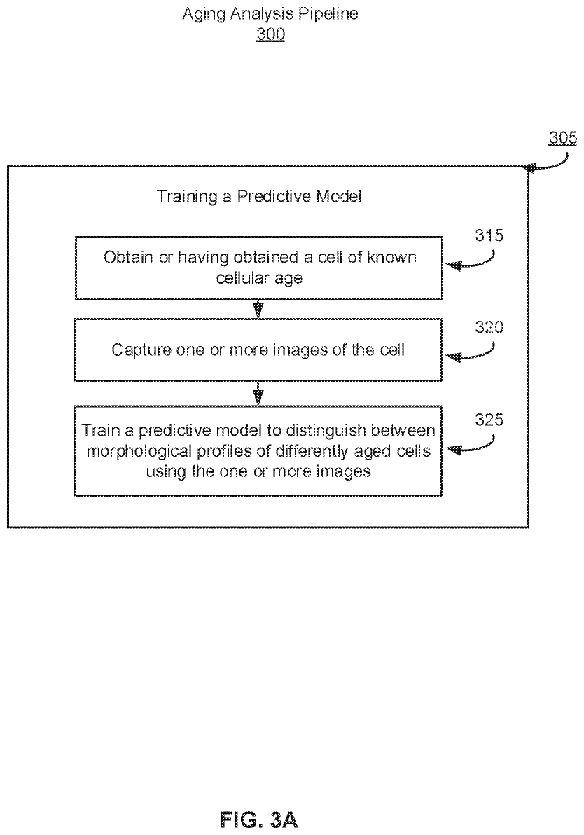

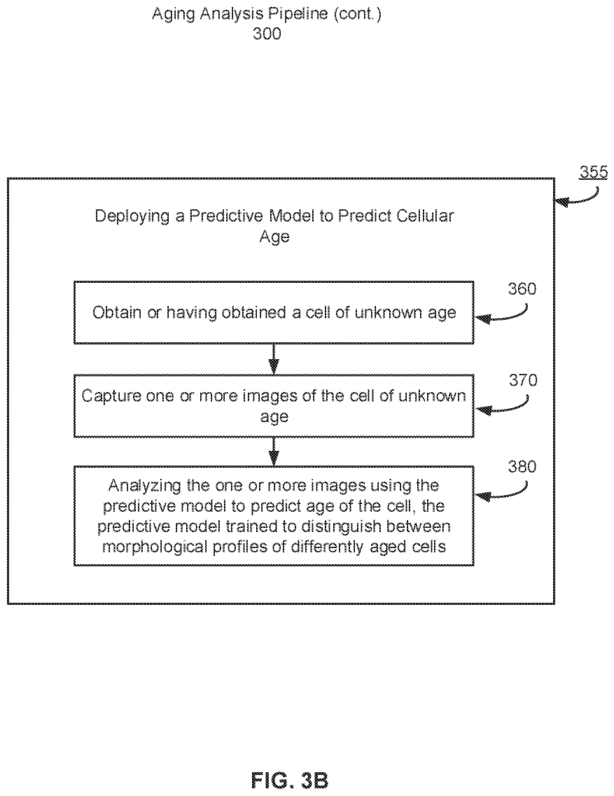

Methods disclosed herein describe the aging analysis pipeline. FIG. 3A is a flow process for training a predictive model for the aging analysis pipeline, in accordance with an embodiment. Furthermore, FIG. 3B is a flow process for deploying a predictive model for the aging analysis pipeline, in accordance with an embodiment.

Generally, the aging analysis pipeline 300 refers to the deployment of a predictive model for predicting the age of a cell, as is shown in FIG. 3B. In various embodiments, the aging analysis pipeline 300 further refers to the training of a predictive model as is shown in FIG. 3A. Thus, although the description below may refer to the aging analysis pipeline as incorporating both the training and deployment of the predictive model, in various embodiments, the aging analysis pipeline 300 only refers to the deployment of a previously trained predictive model.

Referring first to FIG. 3A, at step 305, the predictive model is trained. Here, the training of the predictive model includes steps 315, 320, and 325. Step 315 involves obtaining or having obtained a cell of known cellular age. For example, the cell may have been obtained from a subject of a known age. Step 320 involves capturing one or more images of the cell. As an example, the cell may have been stained (e.g., with Cell Paint stains) and therefore, the different images of the cell correspond to different fluorescent channels that include fluorescent intensity indicating the cell nuclei, nucleic acids, endoplasmic reticulum, actin/Golgi/plasma membrane, and mitochondria.

Step 325 involves training a predictive model to distinguish between morphological profiles of differently aged cells using the one or more images. In various embodiments, the predictive model constructs a morphological profile that includes values of features extracted from one or more images. In various embodiments, a feature extraction process can be performed on the one or more images of the cell. Thus, extracted features can be included in the morphological profile of the cell. Given the reference ground truth value for the cell (e.g., the known cellular age), the predictive model is trained to improve its prediction of the age of the cell.

Referring now to FIG. 3B, at step 355, a trained predictive model is deployed to predict the cellular age of a cell. Here, the deployment of the predictive model includes steps 360, 370, and 380. Step 360 involves obtaining or having obtained a cell of unknown age. As one example, the cell may be undergoing a quality control check and therefore, is evaluated for its age. As another example, the cell may have been perturbed (e.g., perturbed using a small molecule drug), and therefore, the perturbation caused the cell to alter its behavior corresponding to a different age. Thus, the predictive model is deployed to determine whether the age of the cell has changed due to the perturbation.

Step 370 involves capturing one or more images of the cell of unknown age. As an example, the cell may have been stained (e.g., with Cell Paint stains) and therefore, the different images of the cell correspond to different fluorescent channels that include fluorescent intensity indicating the cell nuclei, nucleic acids, endoplasmic reticulum, actin/Golgi/plasma membrane, and mitochondria.

Step 380 involves analyzing the one or more images using the predictive model to predict the age of the cell. Here, the predictive model was previously trained to distinguish between morphological profiles of differently aged cells. Thus, in some embodiments, the predictive model predicts an age of the cell by comparing the morphological profile of the cell with morphological profiles of cells of known cellular age.

### Methods for Developing Cellular Aging Assays

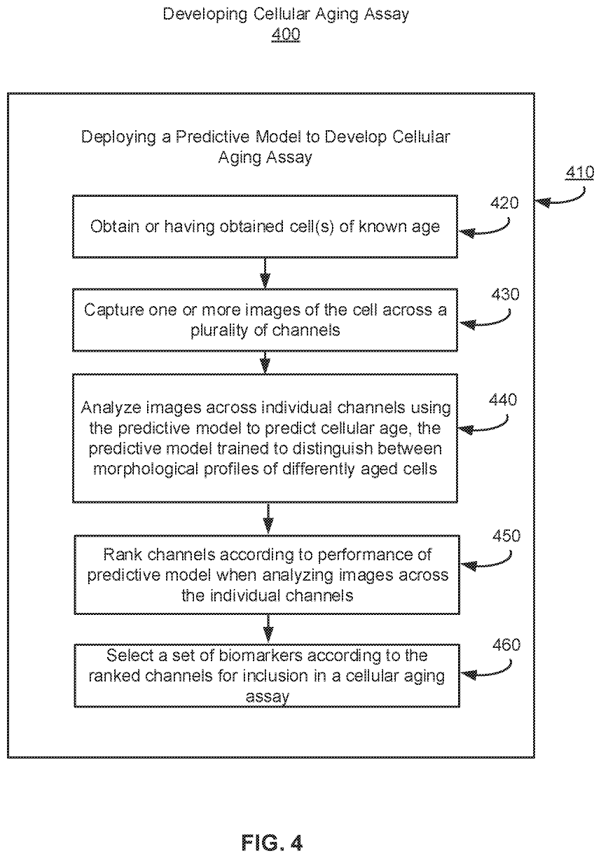

FIG. 4 is a flow process 400 for developing a cellular aging assay by deploying a predictive model, in accordance with an embodiment. For example, the predictive model may, in various embodiments, be trained using the flow process step 305 described in FIG. 3A.

Here, step 410 of deploying a predictive model to develop a cellular aging assay involves steps 420, 430, 440, 450, and 460. Step 420 involves obtaining or having obtained a cell of known age. For example, the cell may have been obtained from a subject of a known age. As another example, the cell may have been previously analyzed by deploying a predictive model (e.g., step 355 shown in FIG. 3B) which predicted a cellular age for the cell.

Step 430 involves capturing one or more images of the cell across a plurality of channels. In various embodiments, each channel comprises signal intensity of a dye that indicates presence of absence of a biomarker. As an example, the cell may have been stained (e.g., with Cell Paint stains) and therefore, the different images of the cell correspond to different fluorescent channels that include fluorescent intensity indicating the cell nuclei, nucleic acids, endoplasmic reticulum, actin/Golgi/plasma membrane, and mitochondria.

Step 440 involves analyzing the one or more images using the predictive model to predict the age of the cell. Here, the predictive model was previously trained to distinguish between morphological profiles of differently aged cells. In particular embodiments, the predictive model is applied to images corresponding to individual channels and the performance of the predictive model is determined based on the analysis of the images for each individual channel. For example, the predictive model is applied to images of a first channel and the performance of the predictive model based on the analysis of the images of the first channel is evaluated. The predictive model is further applied to images of a second channel, and the performance of the predictive model based on the analysis of the images of the second channel is evaluated. Here, the performance of the predictive model is determined according to the reference ground truth e.g., the known age of the cell.

At step 450, the different channels are ranked according to the performance of the predictive model when analyzing images for each of the individual channels. Thus, the top ranked channels are indicative of markers that can be most informative for predicting the age of a cell. At step 460, a set of markers are selected for inclusion in the cellular aging assay, the selected set of markers corresponding to the top ranked channels.

Thus, in subsequent analysis of cells of unknown age, the cells can be stained or labeled for presence or absence of the selected set of biomarkers included in the cellular aging assay. Images captured of cells labeled for the presence of absence of the selected set of biomarkers can be used to further train a predictive model (e.g., train in accordance with step 305 described in FIG. 3A). Thus, the newly developed cellular aging assay can be used to further train and improve the predictive capacity of the predictive model.

### Methods for Determining Modifiers of Cellular Age

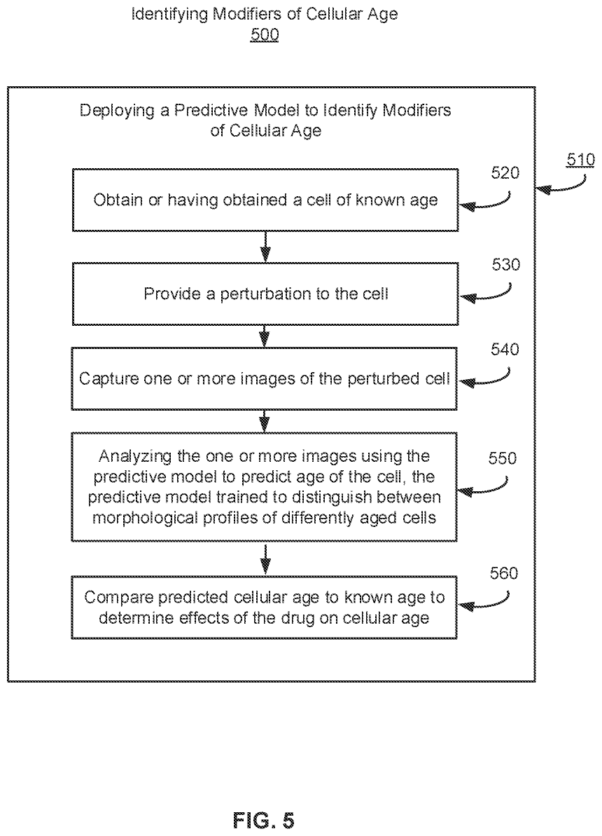

FIG. 5 is a flow process 500 for identifying modifiers of cellular age by deploying a predictive model, in accordance with an embodiment. For example, the predictive model may, in various embodiments, be trained using the flow process step 305 described in FIG. 3A.

Here, step 510 of deploying a predictive model to identify modifiers of cellular age involves steps 520, 530, 540, 550, and 560. Step 520 involves obtaining or having obtained a cell of known age. For example, the cell may have been obtained from a subject of a known age. As another example, the cell may have been previously analyzed by deploying a predictive model (e.g., step 355 shown in FIG. 3B) which predicted a cellular age for the cell.

Step 530 involves providing a perturbation to the cell. For example, the perturbation can be provided to the cell within a well in a well plate (e.g., in a well of a 96 well plate). Here, the provided perturbation may have directed aging or directed rejuvenation effects, which can be manifested by the cell as changes in the cell morphology. Thus, subsequent to providing the perturbation to the cell, the cellular age of the cell may no longer be known.

Step 540 involves capturing one or more images of the perturbed cell. As an example, the cell may have been stained (e.g., with Cell Paint stains) and therefore, the different images of the cell correspond to different fluorescent channels that include fluorescent intensity indicating the cell nuclei, nucleic acids, endoplasmic reticulum, actin/Golgi/plasma membrane, and mitochondria.

Step 550 involves analyzing the one or more images using the predictive model to predict the age of the perturbed cell. Here, the predictive model was previously trained to distinguish between morphological profiles of differently aged cells. Thus, in some embodiments, the predictive model predicts an age of the cell by comparing the morphological profile of the cell with morphological profiles of cells of known cellular age.

Step 560 involves comparing the predicted cellular age to the previous known age of the cell (e.g., prior to perturbation) to determine the effects of the drug on cellular age. For example, if the perturbation caused the cell to exhibit morphological changes that were predicted to be more of an aged phenotype, the perturbation can be characterized as having a directed aging effect on cells. As another example, if the perturbation caused the cell to exhibit morphological changes that were predicted to be a younger phenotype, the perturbation can be characterized as having a directed rejuvenation effect on cells.

### Cells

In various embodiments, the cells (e.g., cells shown in FIG. 1) refer to a single cell. In various embodiments, the cells refer to a population of cells. In various embodiments, the cells refer to multiple populations of cells. The cells can vary in regard to the type of cells (single cell type, mixture of cell types), or culture type (e.g., in vitro 2D culture, in vitro 3D culture, or ex vivo). In various embodiments, the cells include one or more cell types. In various embodiments, the cells are a single cell population with a single cell type. In various embodiments, the cells are stem cells. In various embodiments, the cells are partially differentiated cells. In various embodiments, the cells are terminally differentiated cells. In various embodiments, the cells are somatic cells. In various embodiments, the cells are fibroblasts. In various embodiments, the cells include one or more of stem cells, partially differentiated cells, terminally differentiated cells, somatic cells, or fibroblasts.

In various embodiments, the cells (e.g., cells 105 shown in FIG. 1) are of a single age. In one aspect, the cells are donated from a subject of a particular age. In one aspect, the cells originate from a subject of a particular age. In one aspect, the cells are reprogrammed to exhibit a morphology profile that corresponds to a subject of a particular age. In various embodiments, a subject may be any one of a young subject (e.g., less than 20 years old), a middle-aged subject (e.g., between 20 and 60 years old), or an old subject (e.g., greater than 60 years old). In various embodiments, the subject may be a fetal subject. In various embodiments, the subject may be an individual with Hutchinson-Gilford progeria syndrome (HGPS). In various embodiments, the subject is X years old. In various embodiments, X is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In various embodiments, the cells (e.g., cells 105 shown in FIG. 1) refer to an age-diverse cohort of cells. For example, an age-diverse cohort of cells refers to a mixture of cells obtained from multiple subjects (e.g., human subjects) of differing ages. In various embodiments, the cells need not be donated from a subject, but may be programmed to exhibit morphology profiles that correspond to subjects of a particular age. Thus, an age-diverse cohort of cells may have cells corresponding to a young subject that exhibit a first morphological profile and cells corresponding to the old subject that exhibit a second morphological profile. In various embodiments, the age of the cells are known as they correspond to the age of a corresponding human. In various embodiments, the age of the cells are unknown and therefore, the predictive model system is used to predict the age of the cells. In various embodiments, the ages of the cells are unknown and therefore, the predictive model system can be used to predict the different age of individual cells.

In various embodiments, the cells are seeded and cultured in vitro in a well plate. In various embodiments, the cells are seeded and cultured in any one of a 6 well plate, 12 well plate, 24 ell plate, 48 well plate, 96 well plate, 192 well plate, or 384 well plate. In particular embodiments, the cells 105 are seeded and cultured in a 96 well plate. In various embodiments, the well plates can be clear bottom well plates that enables imaging (e.g., imaging of cell stains e.g., cell stain 150 shown in FIG. 1).

In various embodiments, different cells are seeded in an in vitro well plate. For example, cells that correspond to the same age can be seeded within a single well in a well plate. For example, cells that correspond to the same age can be seeded within a single well in a well plate. Thus, a well plate can have different individual wells of cells corresponding to different ages. For example, a single well plate can hold a cell line corresponding to a young subject in a first well, a cell line corresponding to a middle-aged subject in a second well, and a cell line corresponding to an old subject in a third well. Thus, when the high-throughput aging analysis pipeline is implemented, the cells of differing ages within the well plate can be imaged simultaneously and processed in parallel.

### Cell Stains

Generally, cells are treated with one or more cell stains or dyes (e.g., cell stains 150 shown in FIG. 1) for purposes of visualizing one or more aspects of cells that can be informative for determining the age of the cells. In particular embodiments, cell stains include fluorescent dyes, such as fluorescent antibody dyes that target biomarkers that represent known aging hallmarks. In various embodiments, cells are treated with one fluorescent dye. In various embodiments, cells are treated with two fluorescent dyes. In various embodiments, cells are treated with three fluorescent dyes. In various embodiments, cells are treated with four fluorescent dyes. In various embodiments, cells are treated with five fluorescent dyes. In various embodiments, cells are treated with six fluorescent dyes. In various embodiments, the different fluorescent dyes used to treat cells are selected such that the fluorescent signal due to one dye minimally overlaps or does not overlap with the fluorescent signal of another dye. Thus, the fluorescent signals of multiple dyes can be imaged for a single cell.

In some embodiments, cells are treated with multiple antibody dyes, where the antibodies are specific for biomarkers that are located in different locations of the cell. For example, cells can be treated with a first antibody dye that binds to cytosolic markers and further treated with a second antibody dye that binds to nuclear markers. This enables separation of fluorescent signals arising from the multiple dyes by spatially localizing the signal from the differently located dyes.

In various embodiments, cells are treated with Cell Paint stains including stains for one or more of cell nuclei (e.g., DAPI stain), nucleoli and cytoplasmic RNA (e.g., RNA or nucleic acid stain), endoplasmic reticulum (ER stain), actin, Golgi and plasma membrane (AGP stain), and mitochondria (MITO stain). Additionally, detailed protocols of Cell Paint staining are further described in Schiff, L. et al., Deep Learning and automated Cell Painting reveal Parkinson's disease-specific signatures in primary patient fibroblasts, bioRxiv 2020.11.13.380576, which is hereby incorporated by reference in its entirety.

Methods disclosed herein further describe the development of a cellular aging assay which includes implementing a predictive model for identifying markers that are informative for the predictive model's performance. For example, markers that influence the predictive model to generate an accurate prediction can be selected for inclusion in a cellular aging assay. Thus, cells can be processed in accordance with the cellular aging assay by staining the cells using dyes indicative of presence or absence of the selected markers. Through these methods, such a cellular aging assay can include biomarkers that are not traditionally recognized to be associated with aging.

### Perturbations

One or more perturbations (e.g., perturbation 160 shown in FIG. 1) can be provided to cells. In various embodiments, a perturbation can be a small molecule drug from a library of small molecule drugs. In various embodiments, a perturbation is a drug or compound that is known to have age-modifying effects, examples of which include rapamycin and senolytics which have been shown to have anti-aging effects. In various embodiments, a perturbation is a drug that affects epigenetic modifications of a cell. In various embodiments, the library of small molecule drugs is a library of small molecule epigenetic modifiers.

In various embodiments, a perturbation is provided to cells that are seeded and cultured within a well in a well plate. In particular embodiments, a perturbation is provided to cells within a well through an automated, high-throughput process.

In various embodiments, a perturbation is applied to cells at a concentration between 1-50 μM. In various embodiments, a perturbation is applied to cells at a concentration between 5-25 μM. In various embodiments, a perturbation is applied to cells at a concentration between 10-15 μM. In various embodiments, a perturbation is applied to cells at a concentration of about 1 μM. In various embodiments, a perturbation is applied to cells at a concentration of about 5 μM. In various embodiments, a perturbation is applied to cells at a concentration of about 10 μM. In various embodiments, a perturbation is applied to cells at a concentration of about 15 μM. In various embodiments, a perturbation is applied to cells at a concentration of about 20 μM. In various embodiments, a perturbation is applied to cells at a concentration of about 25 μM. In various embodiments, a perturbation is applied to cells at a concentration of about 40 μM. In various embodiments, a perturbation is applied to cells at a concentration of about 50 μM.

### Imaging Device

The imaging device (e.g., imaging device 120 shown in FIG. 1) captures one or more images of the cells which are analyzed by the predictive model system 130. The cells may be cultured in an e.g., in vitro 2D culture, in vitro 3D culture, or ex vivo. Generally, the imaging device is capable of capturing signal intensity from dyes (e.g., cell stains 150) that have been applied to the cells. Therefore, the imaging device captures one or more images of the cells including signal intensity originating from the dyes. In particular embodiments, the dyes are fluorescent dyes and therefore, the imaging device captures fluorescent signal intensity from the dyes. In various embodiments, the imaging device is any one of a fluorescence microscope, confocal microscope, or two-photon microscope.

In various embodiments, the imaging device captures images across multiple fluorescent channels, thereby delineating the fluorescent signal intensity that is present in each image. In one scenario, the imaging device captures images across at least 2 fluorescent channels. In one scenario, the imaging device captures images across at least 3 fluorescent channels. In one scenario, the imaging device captures images across at least 4 fluorescent channels. In one scenario, the imaging device captures images across at least 5 fluorescent channels.

In various embodiments, the imaging device captures one or more images per well in a well plate that includes the cells. In various embodiments, the imaging device captures at least 10 tiles per well in the well plates. In various embodiments, the imaging device captures at least 15 tiles per well in the well plates. In various embodiments, the imaging device captures at least 20 tiles per well in the well plates. In various embodiments, the imaging device captures at least 25 tiles per well in the well plates. In various embodiments, the imaging device captures at least 30 tiles per well in the well plates. In various embodiments, the imaging device captures at least 35 tiles per well in the well plates. In various embodiments, the imaging device captures at least 40 tiles per well in the well plates. In various embodiments, the imaging device captures at least 45 tiles per well in the well plates. In various embodiments, the imaging device captures at least 50 tiles per well in the well plates. In various embodiments, the imaging device captures at least 75 tiles per well in the well plates. In various embodiments, the imaging device captures at least 100 tiles per well in the well plates. Therefore, in various embodiments, the imaging device captures numerous images per well plate. For example, the imaging device can capture at least 100 images, at least 1,000 images, or at least 10,000 images from a well plate. In various embodiments, when the high-throughput cellular aging system 140 is implemented over numerous well plates and cell lines, at least 100 images, at least 1,000 images, at least 10,000 images, at least 100,000 images, or at least 1,000,000 images are captured for subsequent analysis.

In various embodiments, imaging device may capture images of cells over various time periods. For example, the imaging device may capture a first image of cells at a first timepoint and subsequently capture a second image of cells at a second timepoint. In various embodiments, the imaging device may capture a time lapse of cells over multiple time points (e.g., over hours, over days, or over weeks). Capturing images of cells at different time points enables the tracking of cell behavior, such as cell mobility, which can be informative for predicting the ages of different cells. In various embodiments, to capture images of cells across different time points, the imaging device may include a platform for housing the cells during imaging, such that the viability of the cultured cells are not impacted during imaging. In various embodiments, the imaging device may have a platform that enables control over the environment conditions (e.g., Oz or CO₂ content, humidity, temperature, and pH) that are exposed to the cells, thereby enabling live cell imaging.

System and/or Computer Embodiments

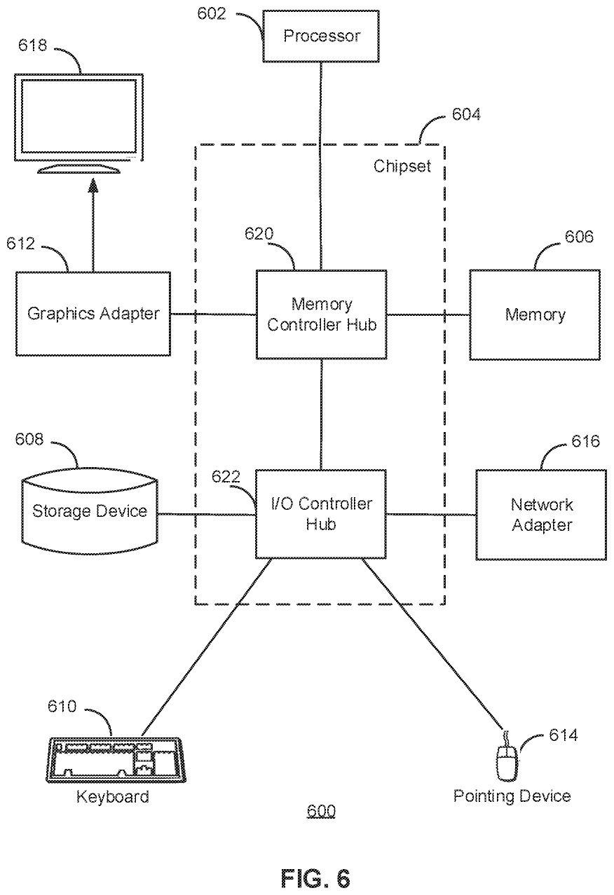

FIG. 6 depicts an example computing device 600 for implementing system and methods described in reference to FIGS. 1-5. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like. In various embodiments, the computing device 600 can operate as the predictive model system 130 shown in FIG. 1 (or a portion of the predictive model system 130). Thus, the computing device 600 may train and/or deploy predictive models for predicting age of cells.

In some embodiments, the computing device 600 includes at least one processor 602 coupled to a chipset 604. The chipset 604 includes a memory controller hub 620 and an input/output (I/O) controller hub 622. A memory 606 and a graphics adapter 612 are coupled to the memory controller hub 620, and a display 618 is coupled to the graphics adapter 612. A storage device 608, an input interface 614, and network adapter 616 are coupled to the I/O controller hub 622. Other embodiments of the computing device 600 have different architectures.

The storage device 608 is a non-transitory computer-readable storage medium such as a hard drive, compact disk read-only memory (CD-ROM), DVD, or a solid-state memory device. The memory 606 holds instructions and data used by the processor 602. The input interface 614 is a touch-screen interface, a mouse, track ball, or other type of input interface, a keyboard, or some combination thereof, and is used to input data into the computing device 600. In some embodiments, the computing device 600 may be configured to receive input (e.g., commands) from the input interface 614 via gestures from the user. The graphics adapter 612 displays images and other information on the display 618. The network adapter 616 couples the computing device 600 to one or more computer networks.

The computing device 600 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 608, loaded into the memory 606, and executed by the processor 602.

The types of computing devices 600 can vary from the embodiments described herein. For example, the computing device 600 can lack some of the components described above, such as graphics adapters 612, input interface 614, and displays 618. In some embodiments, a computing device 600 can include a processor 602 for executing instructions stored on a memory 606.

The methods disclosed herein can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

### Additional Embodiments

Disclosed herein is a method of performing an automated assay comprising: a) providing an age-diverse cohort of cells having a mixture of cell lines of different ages, wherein each of the cell lines has a known cellular age; b) culturing each of the cell lines on an automated platform in a high throughput format and performing cell painting morphological profiling of each of the cell lines, wherein the cell painting morphological profiling comprises generating a plurality of images of each cell line over time; c) analyzing the plurality of images to identify sub-cohorts of cells, each of the sub-cohorts having a different phenotypic cellular age profile thereby classifying sub-cohorts of known cellular age; d) performing cell painting morphological profiling using a putative cell of unknown age; and e) determining the cellular age of the putative cell by comparing the cell painting morphological profile of the putative cell line with that of a sub-cohort of known cellular age. In various embodiments, wherein determining is performed using machine learning and/or deep learning. In various embodiments, wherein the cohort of cells comprises somatic cells. In various embodiments, wherein the somatic cells are fibroblasts. In various embodiments, the cohort of cells comprises one or more of stem cells, partially differentiated cells and terminally differentiated cells. In various embodiments, comprises classifying the putative cell as being a stem cell, partially differentiated cell or terminally differentiated cell. In various embodiments, wherein the plurality of images comprises greater than 100, 1,000 or 1,000,000 images. In various embodiments, the different phenotypic cellular age profiles comprise cell morphological features. In various embodiments, cell morphological features are determined via fluorescently labeled biomarkers.

Additionally disclosed herein is a method of generating a computer database of stored phenotypic cellular age profiles for a plurality of cell lines, the method comprising: a) providing an age-diverse cohort of cells having a mixture of cell lines of different ages, wherein each of the cell lines has a known cellular age; b) culturing each of the cell lines on an automated platform in a high throughput format and performing cell painting morphological profiling of each of the cell lines, wherein the cell painting morphological profiling comprises generating a plurality of images of each cell line over time: c) analyzing the plurality of images to identify sub-cohorts of cells, each of the sub-cohorts having a different phenotypic cellular age profile thereby classifying sub-cohorts of known cellular age; and d) storing the phenotypic cellular age profile of each sub-cohort on a non-transitory computer readable medium. In various embodiments, analyzing is performed using machine learning and/or deep learning. In various embodiments, the cohort of cells comprises somatic cells. In various embodiments, the somatic cells are fibroblasts. In various embodiments, the cohort of cells comprises one or more of stem cells, partially differentiated cells and terminally differentiated cells. In various embodiments, analyzing comprises classifying the sub-cohorts as being stem cells, partially differentiated cells or terminally differentiated cells. In various embodiments, the plurality of images comprises greater than 100, 1,000 or 1,000,000 images. In various embodiments, the different phenotypic cellular age profiles comprise cell morphological features. In various embodiments, the cell morphological features are determined via fluorescently labeled biomarkers. In various embodiments, the high throughput format comprises an automated system or platform, such as an automated array.

Additionally disclosed herein is a method for determining cellular age comprising: a) performing cell painting morphological profiling of a putative cell of unknown age, wherein the cell painting morphological profiling comprises generating a plurality of images of the putative cell over time: b) generating a phenotypic cellular age profile for the putative cell; c) comparing the phenotypic cellular age profile for the putative cell to the phenotypic cellular age profiles with a database of stored phenotypic cellular age profiles from cell lines of known age using machine learning and/or deep learning to perform the comparison; and d) determining the cellular age of the putative cell by the comparison of (c) using machine learning and/or deep learning, wherein the cellular age of the putative cell is the same as a cell line of the database with a similar phenotypic cellular age profile, thereby determining the cellular age of the putative cell. In various embodiments, the cell lines of known age comprise somatic cells. In various embodiments, the somatic cells are fibroblasts. In various embodiments, the cell lines of known age comprise one or more of stem cells, partially differentiated cells and terminally differentiated cells. In various embodiments, determining comprises classifying the putative cell as being a stem cell, partially differentiated cell or terminally differentiated cell. In various embodiments, the plurality of images comprises greater than 100, 1,000 or 1,000,000 images. In various embodiments, the phenotypic cellular age profile comprises cell morphological features. In various embodiments, cell morphological features are determined via fluorescently labeled biomarkers. In various embodiments, the cell painting morphological profiling is performed using a high throughput format. In various embodiments, the high throughput format comprises an automated array.

Additionally disclosed herein is a method of performing an automated screening assay comprising: a) providing a cell of a cell line having defined cellular age and morphological characteristics; b) culturing the cell on an automated platform in a high throughput format; c) contacting the cell with a test agent; d) performing cell painting morphological profiling of the cell, wherein the cell painting morphological profiling comprises generating a plurality of images of the cell over time; and e) analyzing the plurality of images to determine whether the test agent alters cellular aging thereby identifying the test agent as an agent that alters cellular aging. In various embodiments, analyzing is performed using machine learning and/or deep learning. In various embodiments, analyzing comprises extracting fixed features of the cell from the plurality of images and comparing the extracted fixed features over time. In various embodiments, the cell is a somatic cell. In various embodiments, the somatic cell is a fibroblast. In various embodiments, the cell is a stem cell, partially differentiated cell or terminally differentiated cells. In various embodiments, the plurality of images comprises greater than 100, 1,000 or 1,000,000 images. In various embodiments, analyzing comprises comparing cell morphological features. In various embodiments, the cell morphological features are determined via fluorescently labeled biomarkers. In various embodiments, the high throughput format comprises an automated system or platform, such as an automated array.

Additionally disclosed herein is a method for performing an automated deep learning profiling of a plurality of cells comprising: a) providing a cohort of cells having a mixture of cell lines of different ages and/or different cell types; b) culturing each of the cell lines on an automated platform in a high throughput format and performing morphological profiling of each of the cell lines, wherein the morphological profiling comprises generating a plurality of images of each cell line over time; c) analyzing the plurality of images to identify sub-cohorts of cells having different morphological features using machine learning and/or deep learning; d) classifying the sub-cohorts of cells by age and/or cell type using machine learning and/or deep learning; and, optionally e) isolating individual sub-cohorts of cells; wherein (a)-(e) are automated, thereby performing deep learning profiling of a plurality of cells. In various embodiments, the cohort of cells comprises somatic cells. In various embodiments, the somatic cells are fibroblasts. In various embodiments, the cohort of cells comprises one or more of stem cells, partially differentiated cells and terminally differentiated cells. In various embodiments, classifying comprises classifying the sub-cohorts as being stem cells, partially differentiated cells or terminally differentiated cells. In various embodiments, the plurality of images comprises greater than 100, 1,000 or 1,000,000 images. In various embodiments, different morphological features are determined via fluorescently labeled biomarkers. In various embodiments, the high throughput format comprises an automated array.

Additionally disclosed herein is a method for performing an automated assay comprising: a) providing a cohort of cells having a mixture of cell lines of different ages and/or different cell types; b) culturing each of the cell lines on an automated platform in a high throughput format and performing vector profiling of each of the cell lines, wherein the vector profiling comprises generating a plurality of images of each cell line over time; c) analyzing the plurality of images to identify sub-cohorts of cells having different cellular motility using machine learning and/or deep learning; d) classifying the sub-cohorts of cells by age and/or cell type using machine learning and/or deep learning; and, optionally e) isolating individual sub-cohorts of cells: wherein (a)-(e) are automated, thereby performing an automated assay. In various embodiments, the cohort of cells comprises somatic cells. In various embodiments, the somatic cells are fibroblasts. In various embodiments, the cohort of cells comprises one or more of stem cells, partially differentiated cells and terminally differentiated cells. In various embodiments, classifying comprises classifying the sub-cohorts as being stem cells, partially differentiated cells or terminally differentiated cells. In various embodiments, the plurality of images comprises greater than 100, 1,000 or 1,000,000 images. In various embodiments, different morphological features are determined via fluorescently labeled biomarkers. In various embodiments, the high throughput format comprises an automated array. In various embodiments, analyzing comprises comparing the movement of cells over time to determine cellular motility.

Additionally disclosed herein is a computer readable medium or media having stored thereon computer executable commands for performing any of the methods disclosed herein.

### EXAMPLES

### Example 1: Example Aging Analysis Pipeline

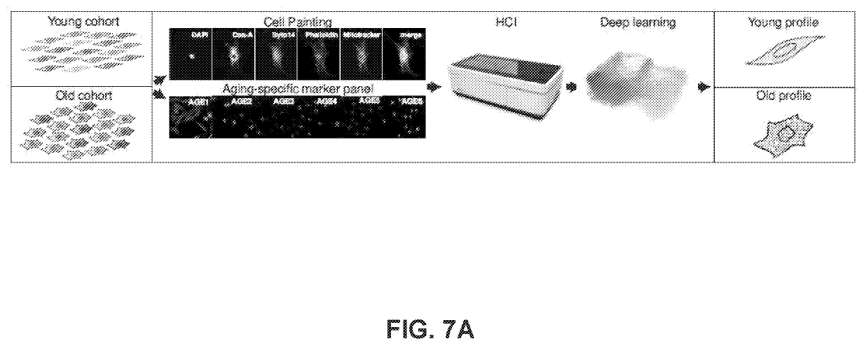

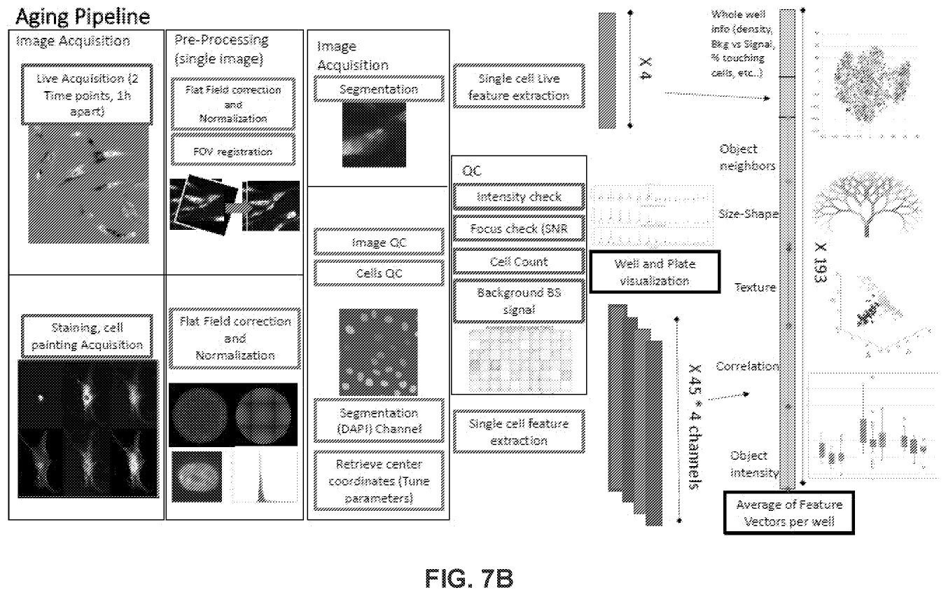

FIG. 7A depicts an example aging analysis pipeline. For example, cohorts of cells (e.g., young and/or old cohorts of cells, or cohorts of cells of varying ages) underwent cell painting morphological profiling to generate a plurality of images (e.g., different images including different cells and/or corresponding to different channels). The plurality of images were used to train a predictive model. As described in the examples, below, the predictive model could be structured as any of a random forest, regression model, or neural network. Thus, predictive models were trained to predict whether cells exhibited a “young profile” or an “old profile.”

FIG. 7B depicts an example aging analysis pipeline in further detail. For example, FIG. 7B depicts in further detail the in silico steps for processing images of cells and training a predictive model using features extracted from the images. In particular, FIG. 7B shows the steps of image acquisition and cell painting acquisition, followed by processing of the images including correction (e.g., flat field correction), normalization, and field of view registration. Additional steps include segmentation of cells according to nuclei staining (e.g., DAPI) as well as quality control checks (e.g., intensity check, focus check, cell count, and background signal analysis). Images then underwent single cell feature extraction. Extracted features include features from a whole well (e.g., density, background versus signal ratio, percentage of touching cells), object neighbor features, cell size features, cell shape features, texture features, correlation features, and object intensity features.

The methodology of an example aging analysis pipeline is described here in further detail. Cells are thawed, propagated, and reseeded into assay plates using existing automation infrastructure. Hardware included in the automation infrastructure is integrated into custom-designed and custom-built “workcells”. Liquid Handlers include Star (multiple), Hamilton including both 96 and 384 pipetting heads and Lynx, Dynamic Devices. Other Robotics include: PF400, Precise Robotics (robotic arm), GX, PAA Robotics (robotic arm), VSPIN, Agilent (centrifuge), LabElite, Hamilton (tube decapper), and Cytomat, ThermoFisher (incubator). Imagers include Celigo, Nexcelom, Opera Phenix, Perkin Elmer, and Ti2, Nikon. Custom software has been written to control the robotic arms, integration with the Phenix imager as well as custom integrations with our centrifuges and incubators. All data is stored/tracked using the NYSCF Websuite/AppSuite.

This cohort also includes samples from Hutchinson-Gilford progeria syndrome (HGPS) patients as positive aging controls. Cell lines are seeded at 3000 cells/well in 96-well format in 12 replicates. Cells are grown following seeding for X days before being stained. Detailed protocols of staining are further described in Schiff, L. et al., Deep Learning and automated Cell Painting reveal Parkinson's disease-specific signatures in primary patient fibroblasts, bioRxiv 2020.11.13.380576, which is hereby incorporated by reference in its entirety. Following staining cells are imaged using the Ti2 or Phenix imager. Up to 80 fields are imaged on an Opera Phenix at 40× magnification in 5 fluorescent channels+brightfield, capturing ˜2000 cells/well. Images undergo single cell segmentation, resulting in approximately 140,000 images/subject and a total of >11 million images per batch of experiments.

For cell mobility assays, cell mobility is captured by a time-lapse imaging assay where 2 images of cultures stained with nuclear and membrane dyes are taken at few hours apart. The nuclear marker allows to segment the cells and register them to the following acquisition, and to identify the displacement of the center of the cells. The membrane stain is needed to identify the micromovements of the peripheral part of the cells. From this acquisition, another portion of the vector is calculated, that includes displacement, amount of new area covered by the cell, amount of area not covered by the cell any longer, overall area difference, and the like. The vector is then analyzed using different types of algorithms and on well basis (by averaging the vectors of single cells in each well), or on single cell basis.

### Example 2: Random Forest Predictive Model Differentiates Cells According to Age

A pilot experiment involving the implementation of a random forest predictive was performed in accordance with the methodology described in Example 1. Specifically, the pilot experiment involved seeding twelve 96-well plates in 2 different layouts, with 30 cell lines and double replicates per plate. 10 cell lines originated from young donors, 10 from old ones, 4 from middle-aged, 4 were embryonic, and 2 belonged to patients affected by progeria. 3 plates were reserved for live imaging, and 8 for the staining panel. As a preliminary result, cell motility and cell morphology were analyzed separately. Cell motility was analyzed to demonstrate that the cells were showing a different motility behavior according to their age. Here, the experiment was performed by analyzing the displacement of the centroid of morphologically labeled cells between two acquisitions spaced one hour apart.

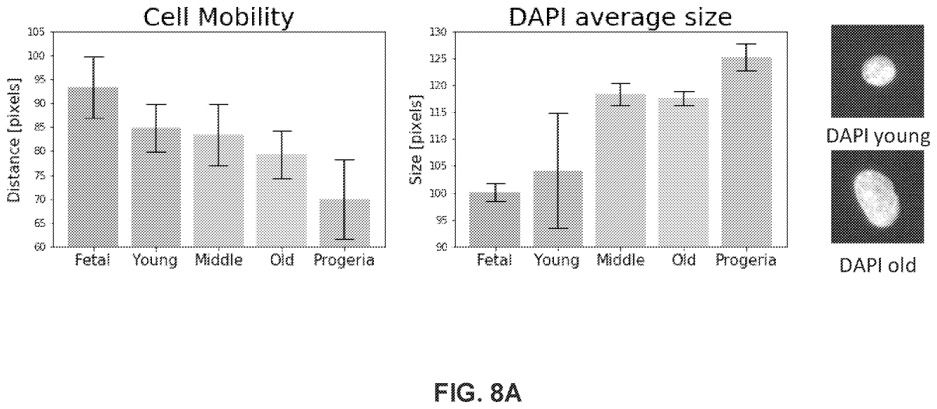

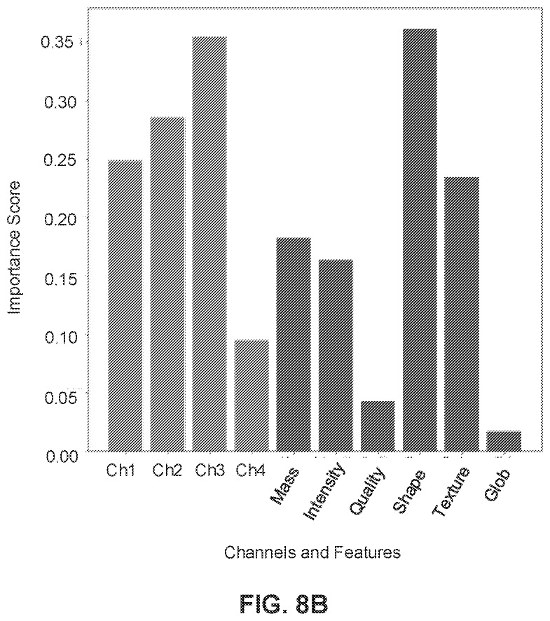

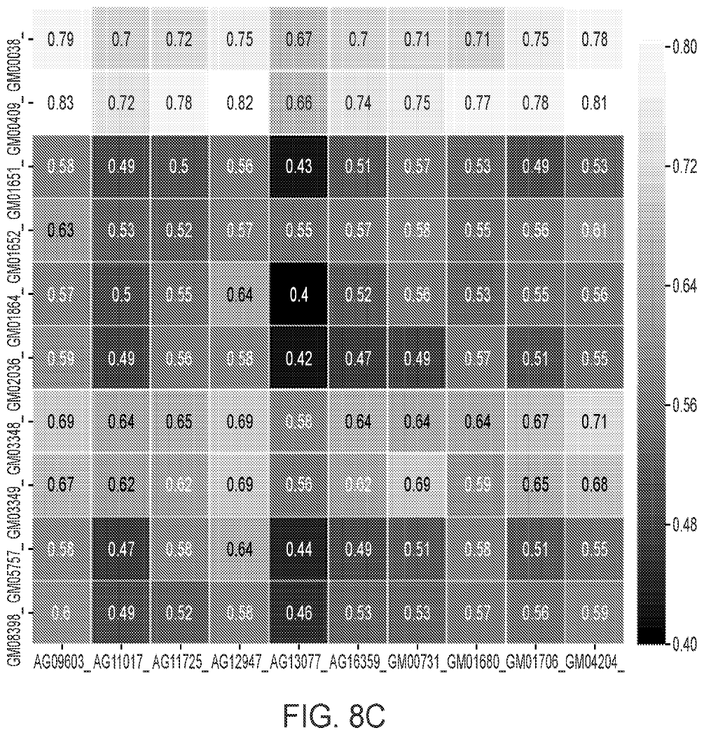

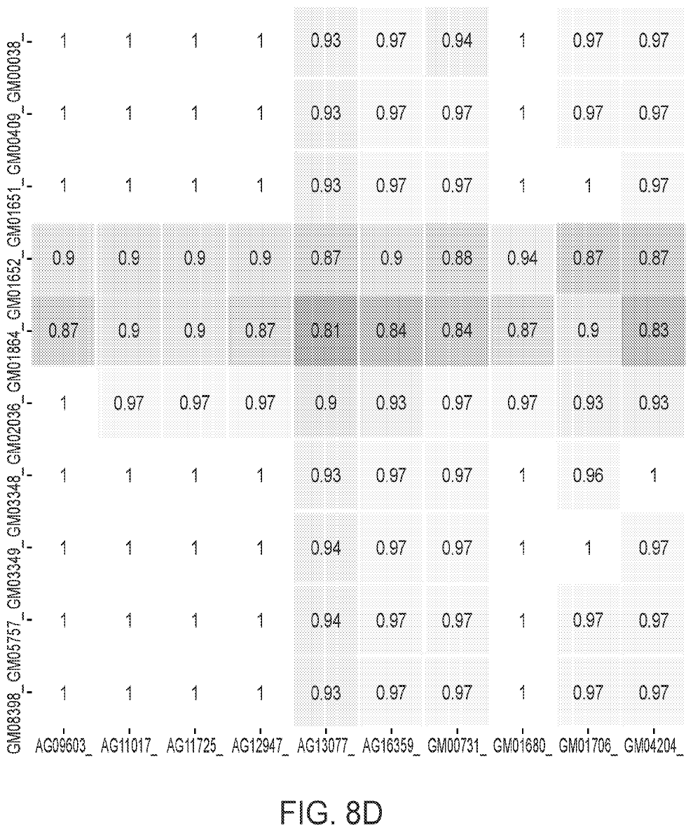

FIG. 8A shows quantitative phenotypic differences across fibroblast cell lines of different ages. Specifically, FIG. 8A shows quantitative phenotypic differences among 30 fibroblast lines from age-diverse donors including fetal cells, young (between 7-13 years of age), middle (31-51 years of age), old (71-96 years of age) and positive control progeria donors. The left panel of FIG. 8A shows average linear displacement of 5000 live cells per donor imaged over 1 hour. The middle panel of FIG. 8A shows average size of nuclei across ˜44K cells per donor. The right panel of FIG. 8A shows images of representative DAPI stained nuclei of young and old fibroblasts.

As shown in the left panel of FIG. 8A, there exists a correlation between age and cell motility, with the more motile cells being the youngest or in fetal stage, and less motile cells being older or progeria. The remaining 8 plates resulted in 49 images per well, 4 channel each. As a first vector calculation, 158 different features were computed per cell. Feeding the computed vectors into a random forest algorithm resulted in an accuracy of 90% on a per-cell basis analysis, and of 96.6% on a well basis, when young and old cell lines were tested. The algorithm was trained on 70% of the data and tested on randomly selected samples.

FIG. 8B shows the sum of the importance score for each major category of the vector, and similarly for the channels. Ch1 to 4 are respectively ConcavallinA, Mitotracker, DAPI, and AGP staining. The feature categories are mass (e.g., how much the morphology is compact or dense), intensity (e.g., the brightness of the segmented region), quality (e.g., signal to noise ratio and focus), shape (e.g., dimension and roundness), texture and global (e.g., related to the surroundings, so how many cells are in the wells, how close the cell is to another).

To ensure that the observed results were not driven by overfitting, entire cell lines were removed from the training set and the accuracy score was then investigated. FIG. 8C demonstrates a matrix showing the accuracy of the random forest classifier when entire cell lines were removed from the training set in a single cell analysis. FIG. 8D demonstrates a matrix showing the accuracy of the random forest classifier when entire cell lines were removed from the training set in a per-well analysis. Generally, the predictive model remained predictive (most of the cell lines were over 90% of accuracy) when young and/or old cell lines were selectively removed from the training set. Specifically, the y-axis in the matrices shown in FIGS. 8C and 8D refer to the removed young cell line and the x-axis in the matrices shown in FIGS. 8C and 8D refer to the removed old cell line. Additionally, the same model was used to calculate the accuracy score on young and old paired cell lines. The performances of the algorithm showed again a very high accuracy, reflecting the phenotypic difference between young and old primary fibroblasts.

### Example 3: Predictive Regression Model Differentiates Cells According to Age

An experiment involving the implementation of a predictive regression model was performed in accordance with the methodology described in Example 1. Here, the goal was to show that a trained regression model (trained on different training datasets) can accurately predict the age of cells of unknown age.

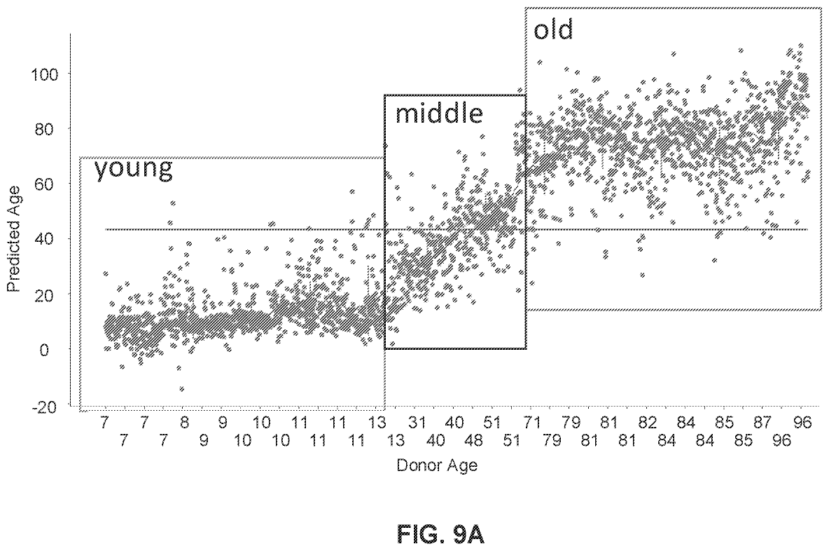

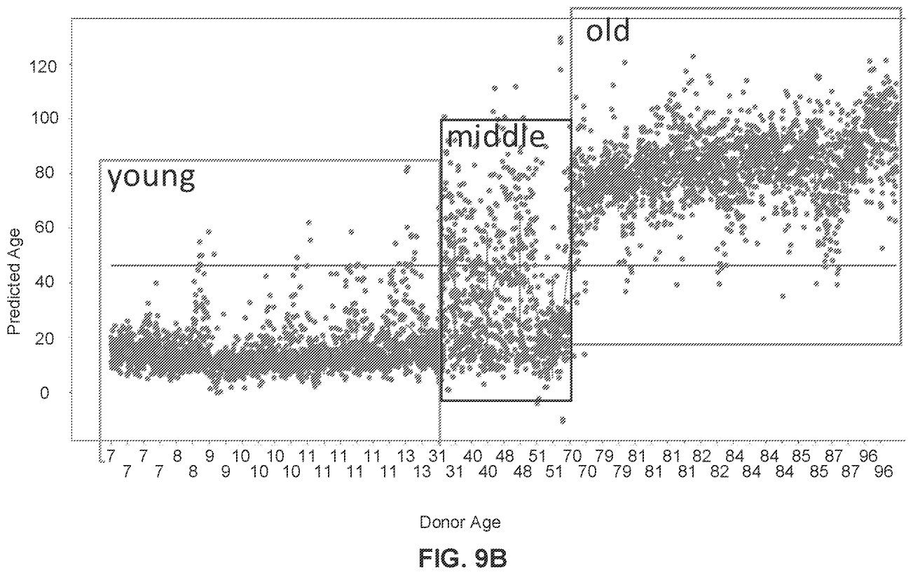

FIG. 9A depicts the predicted age determined by a regression model trained at the single cell level using young, middle aged, and old cells. FIG. 9B depicts the predicted age determined by a regression model trained at the single cell level using young and old cells. As observed in both FIGS. 9A and 9B, the regression model was able to accurately distinguish between young cells and old cells. The regression model trained on young, middle-aged, and old cells (as shown in FIG. 9A) was further able to generally distinguish middle-aged cells from young and old cells. Notably, even the regression model trained only on young cells and old cells was able to predict middle-aged cells, even without having seen such middle-aged cells before. This demonstrates that there is likely a continuum of cellular morphological features that represent cells at different ages and the predictive model is able to identify such morphological features.

### Example 4: Predictive Neural Network Differentiates Cells According to Age

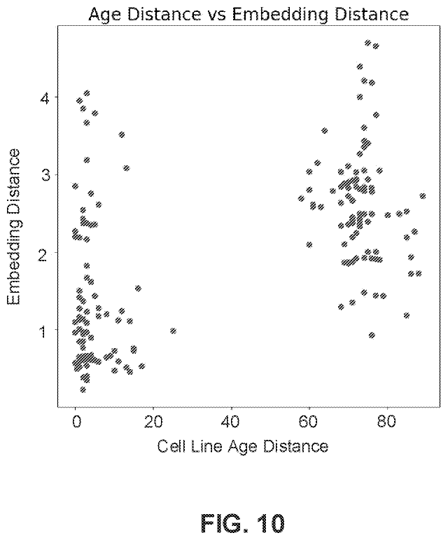

In many scenarios, deep learning outperforms other image analysis approaches. Here, a deep learning algorithm was applied that reduces each image to a set of embeddings that can be plotted in a multidimensional vector space. Images from 3/4 of the subjects was used as part of a training dataset for training a CNN for binary age classification (young: <20 vs old: >80). The remaining data was used as a test set. Two commonly used CNN architectures were implemented including ResNet50 and Inception-v4. All CNN implementations and training were conducted using the Python 3 environment, incorporating elements of the machine-learning libraries Keras and Tensorflow. First, a weakly-supervised representation-learning approach was employed using single-cell images labelled as either young (<10 years) or old (>70 years). In this approach, high-dimensional embeddings are extracted from the neural network's penultimate layer which are used comparatively by calculating the absolute Euclidean distance between the mean embeddings for biological samples as a measure of phenotypic distance. FIG. 10 shows a embedding distance versus actual cell line age distance. Here, FIG. 10 shows that older cells (as indicated by a greater cell line age distance) generally correlate with a higher distance within the embedding (as indicated by a greater embedding distance), thereby indicating that the embedding can successfully distinguish between cells of differing ages.

### Example 5: Example Markers for a Cellular Aging Assay

Primary fibroblasts from fetal, young, old and Hutchinson Gilford Progeria Syndrome (HGPS) donors have been profiled in collaboration via total RNA-Seq, enhanced reduced representation bisulfite sequencing (ERRBS) for genome-wide DNA methylation analysis and ATAC-seq for genome-wide chromatin accessibility mapping. These genomic profiling data can be analyzed to generate a fibroblast-specific epigenomic aging signature, which enable molecular validation of candidate age-modifying compounds.

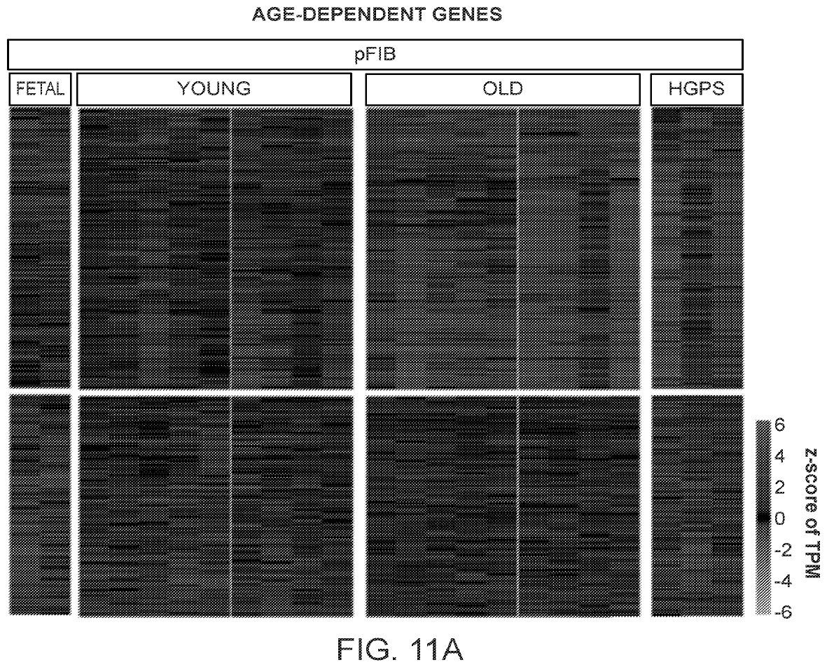

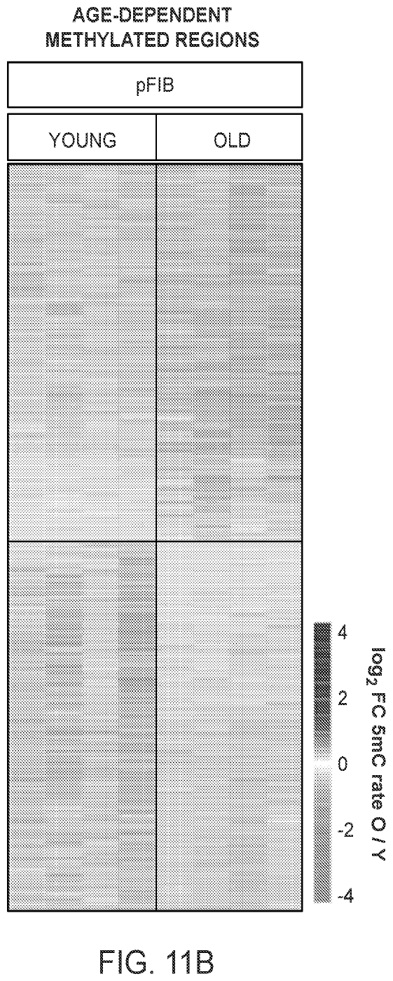

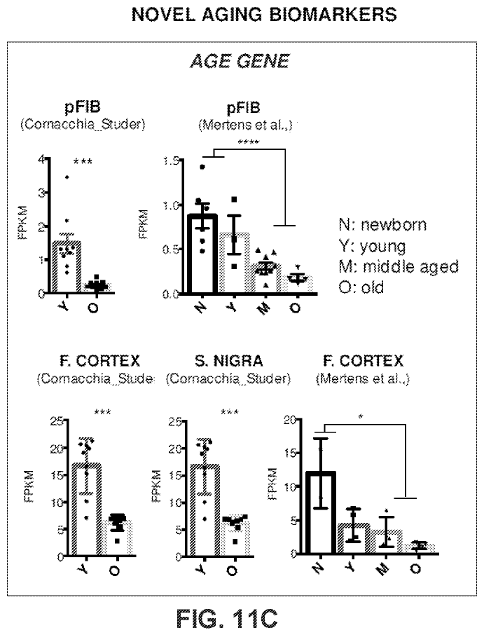

FIGS. 11A-11C show epigenomic age profiling of differently aged primary fibroblasts. Specifically, FIG. 11A shows a heat map of top age-regulated genes. This identifies gene loci that are linearly regulated with age, where expression in older cells resemble progeria (HGPS) primary fibroblasts, collectively generating an initial transcriptomic aging signature. FIG. 11B shows identification of differentially methylated regions in young and old fibroblasts using ERRBS. FIG. 11C shows alignment of RNA-Seq data from fibroblasts and brain in collaboration with published RNA-Seq datasets from fibroblasts and brain identified novel robust aging biomarkers in both tissues. Altogether, FIGS. 11A-11C show candidate genes and biomarkers that may be analyzed using a predictive model according to the flow process described in FIG. 4 and/or included in a cellular aging assay depending on the performance of the predictive model.

These candidate genes and biomarkers are stained in a Cell Painting panel and individually analyzed to determine best predictors of cellular age. To determine the individual contribution of each dye in the original Cell Painting panel to cell age prediction, each channel corresponding to a dye is analyzed individually in silico by applying a predictive model, such as the predictive models described in any of Examples 2-4. The channels are ranked based on their individual ability to predict both binary (<20 and 60> years) and ˜5 binned age ranges.

10 antibody-based markers are chosen that represent known aging hallmarks as well as original molecular markers. 10 aging markers are tested at a time by combining a cytosolic marker and a nuclear marker in each of the 5 channels. The nucleus or cytosol is segmented and masked. Images from each of the 10 segmented classes are fed individually into both algorithms their ability to predict aging is ranked.

The best dyes and antibody markers are combined into an aging-tailored Cell Painting assay. Initially up to 5 pairs of the best performing nuclear and cytosolic markers are combined. Thus, this custom Cell painting assay is used e.g., to reanalyze the 96-cell line cohort described above in Example 1. To assemble the most predictive panel of markers, images from each segmented dye of this profiling run is analyzed individually in silico. Sequential analysis is performed, thereby determining how the predictive power of the panel changes by including the highest ranked dyes, one by one, in the analysis panel.

### Example 6: Predictive Model Differentiates Differentially Perturbed Cells

Among the most potent strategies capable of reversing age-dependent features in single cells as well as entire organisms is somatic cell reprogramming. Recent advancements in epigenetically active drug development and epigenome editing technologies have vastly increased the precision of targeted epigenetic modifications at specific loci or genome-wide, expanding the opportunities for age-reprogramming strategies directed at single cells or whole organisms.

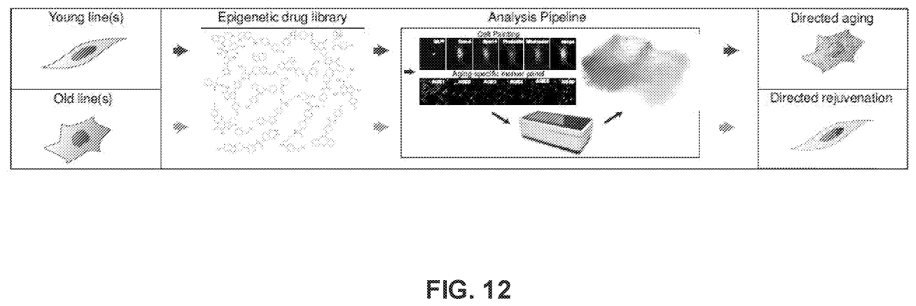

FIG. 12 depicts an example drug screening pipeline. As shown in FIG. 12, the example drug screening pipeline includes the aging analysis pipeline (labeled as “Analysis Pipeline” in FIG. 12) that was described above in Example 1. Thus, the screening pipeline involves perturbing cell cohorts (e.g., young or old cell lines) with a drug from an epigenetic drug library. Perturbed cells then undergo the aging analysis pipeline to determine whether the age of the cells have changed in response to the drug perturbation. This is useful for understanding the effects of particular drugs and whether individual dugs are useful for directed aging or directed rejuvenation.

By applying this pipeline to the Cell Painting assay, which captures images from each cell in 5 fluorescent channels, each cell is represented as a 320-dimensional embedding. These embeddings provide robust, quantitative representations of cellular phenotypes when placed in high-dimensional space where more similar cells are located closer together. These representations are visualized using standard dimensionality reduction techniques such as principal component analysis (PCA) and t-distributed Stochastic Neighbor Embedding (t-SNE). Such visualizations are exceptionally informative in identifying complex morphological drug responses: cells cluster together based on the mechanism of action (MoA) of the compounds they were treated with.

The cell aging and rejuvenating screening assays is performed in parallel using young and old cell lines showing divergent phenotypes. Phenotypic separation between the young and old cell lines are verified and assay parameters are optimized to achieve a Z score greater than 0.5. For the aging rescue upon progeria treatment assay, a young or fetal fibroblast line that shows a representative ‘young’ morphological profile is selected. The fibroblast line undergoes accelerated aging by transfecting with modified RNA to drive overexpression of progeria, the mutant LAMIN A isoform responsible for HGPS. This causes rapid onset of the aging phenotype. The analysis pipeline is implemented to robustly track the onset and progression of aging signatures upon progeria treatment and to determine optimal assay endpoints.

Drug screens are performed using two representative young and aged cell lines in a single-dose, single-replicate per line design. The final screening concentration for all compounds in the library (e.g., 1-50 μM) are selected based on assay parameters, DMSO tolerance, compound solubility, and previous library data. As positive controls, drugs with known age-modifying effects (e.g., rapamycin and senolytics) are used to treat cell lines. Automated procedures are used to thaw, adapt, passage, and seed 1000 cells into each well of a 384-well plate. Aliquots of compound libraries are prepared at desired concentrations using a liquid handling robot. Following 4 days of treatment, the plates are profiled using the automated, fully optimized, Cell Painting phenotyping pipeline described in Example 1. To control for plate-to-plate variability and provide robust controls for phenotypic shifts, DMSO-treated cells from all 4 donors are present in 36 wells in each assay plate. Hit compounds that induce, reverse, or inhibit the onset of aging phenotypes (as predicted by a predictive model e.g., any predictive model described in Examples 2-4) are tested on up to 10 additional young and old lines to determine specificity. Confirmed hits are subjected to a dose-response curve to determine whether full phenotypic reversal is possible and which compounds exhibit the most suppressive effects. Hit validation can be performed via genome-wide methylation analysis to verify pharmacological reversal of age-related epigenetic profiles.

Average feature embeddings are computed for untreated young, aged, and progeria expressing cell lines, establishing an intra-experiment screening phenotype. Average feature embeddings for all cells within each treatment condition are similarly computed. Standard dimensionality reduction (t-SNE) is used to visualize the results in 2D space. The goal of the screens is to identify compounds that shift aged feature embeddings towards those of young controls and vice versa, i.e., a hit would cause feature embeddings for treated aged cells to cluster more closely to young controls than to untreated aged cells. Hit compounds are selected based on a criterion e.g., a standard deviation of 3 or more. The criterion can also depend on assay robustness and the number of total hits.

1-82. (canceled)

83. A method comprising:
obtaining or having obtained a cell;
capturing one or more images of the cell; and
analyzing the one or more images using a predictive model to predict a cellular age of the cell, the predictive model trained to distinguish between morphological profiles of differently aged cells.

84. The method of claim 83, further comprising:
prior to capturing one or more images of the cell, providing a perturbation to the cell;
subsequent to analyzing the one or more images, comparing the predicted cellular age of the cell to an age of the cell known before providing the perturbation; and
based on the comparison, identifying the perturbation as having one of a directed aging effect, directed rejuvenation effect, or no effect.

85. The method of claim 83, wherein analyzing the one or more images using a predictive model comprises separately applying the predictive model to each of the one or more images to predict cellular ages, wherein the method further comprises:
evaluating performances of the predictive model across the predicted cellular ages;
ranking the one or more images according to the evaluated performances of the predictive model across the predicted cellular ages; and
selecting a set of biomarkers corresponding to the ranked channels for inclusion in a cellular aging assay.

86. The method of claim 83, wherein the predictive model is one of a neural network, random forest, or regression model.

87. The method of claim 83, wherein each of the morphological profiles of differently aged cells comprises values of imaging features that define an age of a cell.

88. The method of claim 87, wherein the imaging features comprise one or more of cell features or non-cell features.

89. The method of claim 88, wherein the cell features comprise one or more of cellular shape, cellular size, cellular organelles, object-neighbors features, mass features, intensity features, quality features, texture features, and global features.

90. The method of claim 88, wherein the non-cell features comprise well density features, background versus signal features, and percent of touching cells in a well.

91. The method of claim 88, wherein the cell features are determined via fluorescently labeled biomarkers in the one or more images.

92. The method of claim 83, wherein the morphological profile is an embedding representing a dimensionally reduced representation of values of a layer of the neural network.

93. The method of claim 83, wherein the cellular age of the cell predicted by the predictive model is a classification of at least two categories.

94. The method of claim 83, wherein the cell is one of a stem cell, partially differentiated cell, or terminally differentiated cell.

95. The method of claim 83, wherein the cell is a somatic cell, and the somatic cell is a fibroblast.

96. The method of claim 83, wherein the predictive model is trained by:
obtaining or having obtained a cell of a known cellular age;
capturing one or more images of the cell of the known cellular age; and
using the one or more images of the cell of the known cellular age, training the predictive model to distinguish between morphological profiles of differently aged cells.

97. The method of claim 96, wherein the known cellular age of the cell serves as a reference ground truth for training the predictive model.

98. The method of claim 96, wherein the cell of the known cellular age is one cell in an age-diverse cohort of cells.

99. The method of claim 83, further comprising:
prior to capturing the one or more images of the cdl, staining or having stained the cell using one or more fluorescent dyes,
wherein the one or more fluorescent dyes are Cell Paint dyes for staining one or more of a cell nucleus, cell nucleoli, plasma membrane, cytoplasmic RNA, endoplasmic reticulum, actin, Golgi apparatus, and mitochondria.

100. The method of claim 83, wherein the steps of obtaining the cell and capturing the one or more images of the cell are performed in a high-throughput format using an automated array.

101. A non-transitory computer-readable medium comprising instructions that, when executed by a processor, cause the processor to:
obtain or having obtained one or more images of a cell; and
analyze the one or more images using a predictive model to predict the cellular age of the cell, the predictive model trained to distinguish between morphological profiles of differently aged cells.

102. A method comprising:
obtaining or having obtained a cell;
capturing one or more images of the cell; and
analyzing imaging features derived from the one or more images using a predictive model to predict the cellular age of the cell, the predictive model trained to distinguish between morphological profiles of differently aged cells,
wherein the imaging features comprise cell features and non-cell features, and
wherein the morphological profiles of differently aged cells comprise values of imaging features that define an age of a cell.
